(19) United States
(12) Patent Application Publication
(10) Pub. No.: US 2007/0130653 A1
(43) Pub. Date: Jun. 7, 2007

(54) Methods and compositions for gene silencing

(75) Inventors: David Baulcombe, Norfolk (GB); Enno Krebbers, Ardentown, DE (US); William D. Hitz, Wilmington, DE (US); Kimberly F. Glassman, Ankeny, IA (US); Milo J. Aukerman, Newark, DE (US); Robert Williams, Hockessin, DE (US); Byung-Chun Yoo, Newark, DE (US)

Correspondence Address: ALSTON & BIRD LLP;PIONEER HI-BRED INTERNATIONAL, INC., BANK OF AMERICA PLAZA, 101 SOUTH TRYON STREET, SUITE 4000, CHARLOTTE, NC, 28280-4000, US

(73) Assignees: Pioneer Hi-Bred International, Inc., Johnston, IA (US); E.I. du Pont de Nemours and Company, Wilmington, DE (US); Plant Bioscience Limited, Norwich (GB)

(21) Appl. No.: 11/455,073

(22) Filed: Jun. 16, 2006

Related U.S. Application Data
(60) Provisional application No. 60/691,613, filed on Jun. 17, 2005.
(60) Provisional application No. 60/753,517, filed on Dec. 23, 2005.

(51) Int. Cl. A01H 1/00; C07H 21/02; C12N 15/82; C07H 21/04; C12N 5/04

(52) U.S. Cl. 800/279; 435/419; 435/468; 536/23.1; 800/285

(57) ABSTRACT

Methods and compositions are provided for reducing the level of expression of a target polynucleotide in an organism. The methods and compositions selectively silence the target polynucleotide through the expression of a chimeric polynucleotide comprising the target for a sRNA (the trigger sequence) operably linked to a sequence corresponding to all or part of the gene or genes to be silenced. In this manner, the final target of silencing is an endogenous gene in the organism in which the chimeric polynucleotide is expressed. In a further embodiment, the miRNA target is that of a heterologous miRNA or siRNA, the latter of which is coexpressed in the cells at the appropriate developmental stage to provide silencing of the final target when and where desired. In a further embodiment, the final target may be a gene in a second organism, such as a plant pest, that feeds upon the organism containing the chimeric gene or genes. Compositions further comprise vectors, seeds, grain, cells, and organisms, including plants and plant cells, comprising the chimeric polynucleotide of the invention.

### CROSS-REFERENCE TO RELATED APPLICATIONS

This application claims the benefit of U.S. Provisional Application No. 60/691,613, filed on Jun. 17, 2005 and U.S. Provisional Application No. 60/753,517, filed on Dec. 23, 2005, both of which are hereby incorporated by reference in their entirety.

### FIELD OF THE INVENTION

The present invention relates generally to molecular biology and gene silencing.

### BACKGROUND OF THE INVENTION

In biotechnology, the ability to silence genes is as useful as the ability to express or over express them. In plants it was shown early that transgenic expression of antisense versions of a gene or even extra sense copies of a gene could result in silencing of the endogenous copy of the same gene, albeit at low frequencies (U.S. Pat. No. 5,107,065, Napoli et al. (1990) Plant Cell 2: 279-289 and U.S. Pat. No. 5,231,020, incorporated herein by reference). It was later found that creating constructs with specific configurations, such as hairpin structures (Han et al. (2002) Mol. Genet. Genomics 276:629-35 and Wang et al. (2002) Plant Mol Biol 43:67-82) could increase the efficiency of the process. Only more recently have the mechanisms of the process begun to be understood with the discovery that double-stranded RNA molecules can silence genes and that such molecules underlie various phenomena including co-suppression, antisense suppression, quelling and post transcriptional gene silencing (PTGS). All of these involve a mechanism known as RNA interference (RNAi), which is based on short (20-25 nucleotide) RNA molecules produced by cleavage of longer double stranded RNAs by an enzyme called dicer (Novina and Sharp (2004) Nature 430:161-164 and Baulcombe (2004) Nature 431:356-363). The longer double stranded RNA molecule may be encoded by a gene or result from the action of RNA dependent RNA polymerase on an aberrant RNA which somehow forms a hairpin, resulting in a primer being present or in fact may be a primer-independent process. Depending on the system, due to the presence of different protein factors and possibly the amount of sequence homology, the resulting short molecules may either join a protein complex called RNA-induced silencing complex (RISC) and, converted to a single strand form, guide that complex to the target mRNA which is then cleaved. Alternatively, they may complex with modified forms of RISC or other ribonucleic acid complexes and then simply basepair with the target mRNA, preventing its translation and thus silence expression (Meister and Tuschl (2004) Nature 431:343-349). In either case, the product of the target gene is not produced. RNA interference can also act at the level of transcription (Bartel and Bartel (2003) Plant Physiol. 132:709-717 and Zilberman et al. (2003) Science 299:716-719).

The source of the double stranded RNAs may include viral infection, endogenous genes encoding a transcript capable of folding back on itself, transgenes deliberately designed to result in a transcript capable of folding back on itself, and transgenes that through imprecise integration in the genome inadvertently produce such transcripts. The endogenous genes referred to above may produce specific fragments, called microRNAs (miRNA) (Bartel (2004) Cell 116:281-297), which often play important roles in development and gene regulation. These are considered in more detail below. Longer double stranded molecules, such as those resulting from viral infection or transgene expression, may produce many possible fragments, called short interfering RNAs (siRNA), each of which has the potential to silence a gene with a sequence homologous to the fragment. siRNAs can also be produced from endogenous genes, but their maturation process is different from that of miRNAs. siRNAs more commonly exert their effect through cleavage of their target, while miRNAs often mediate translational inhibition of their target, but siRNAs may act as miRNAs and vice versa (Meister and Tuschl (2004) Nature 431:343-349). In plants, in particular, current evidence suggests that miRNAs more often act through RNA cleavage than via translational inhibition. miRNAs and siRNAs will collectively be referred to as sRNAs (small RNAs).

miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) and miRNA*. The pre-miRNA is a substrate for a form of dicer that removes the miRNA/miRNA* duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Genomic surveys have made possible the identification of the targets of many miRNAs and siRNAs. Both have been shown to play important roles in development. Allen et al. ((2005) Cell 121:207-221) have demonstrated that pathways involving the two interact. Specifically, several examples were found of miRNAs used to mediate the processing of transcripts that contain the precursors for multiple siRNAs. The targets sites may be at the 5′ or 3′ end of the siRNA precursor transcript, and cleavage by the miRNA appears to set the “register” for the dicer enzyme so that the correct siRNAs are produced after RNA dependent RNA polymerase forms the second strand of the precursor. Parizotto et al. ((2004) Genes & Development 18:2237-2242) had previously shown that RNAi could be used to monitor the activity of a miRNA by expressing a chimeric gene including the gene encoding a fluorescent protein operably linked to the target of a miRNA. When the miRNA was present, the mRNA encoded by the transgene was degraded, resulting in a lack of fluorescence. Small RNAs derived from the region upstream of the miRNA target site were detected, and their synthesis was dependent on an RNA dependent RNA polymerase RDR6, also known as SDE1 or SGS2.

In the examples referred to above, the silencing mechanism acts through siRNA or miRNA directed cleavage of a target RNA. However there are related siRNA-directed mechanisms in which the target molecule is DNA or RNA in chromatin and in which the final outcome of the process is suppression of transcription. This RNA-mediated RNA silencing operates at the chromatin level and is associated in plants with DNA methylation and with histone modifications in many organisms. The first evidence for this type of silencing was the discovery in plants that transgene and viral RNAs guide DNA methylation (Wassenegger et al. (1994) Cell 76:567-576; Mette et al. (2000) EMBO J. 19:5194-5201 and Jones et al. (2001) Curr. Biol. 11: 747-757) to specific nucleotide sequences. More recently these findings have been extended by the findings that siRNA-directed DNA methylation in plants is linked to histone modification (Zilberman et al. (2003) Science 299:716-719) and, in fission yeast, that heterochromatin formation at centromere boundaries is associated with siRNAs (Volpe et al. (2002) Science 297:1833-1837). An important role of RNA silencing at the chromatin level is likely in protecting the genome against damage caused by transposons (Lippman and Martienssen (2004) Nature 431:364-370).

The ability to manipulate the gene silencing pathways provides significant advantages in the field of biotechnology. Novel methods and compositions are therefore needed in the art to allow for the targeted silencing of genes.

### BRIEF DESCRIPTION OF THE DRAWINGS

FIG. 1 provides a non-limiting example of a chimeric polynucleotide of the invention. The construct comprises the Basta selectable marker driven by the nos promoter and the 35S promoter driving a chimeric construct comprising the GFP polynucleotide, a silencer sequence (a fragment of the gene to be silenced), and the trigger sequence followed by the terminators of the 35S gene. The four non-limiting genes chosen as genes to be silenced include the chalcone synthase gene (CHS) (reduced expression resulting in a pigmentation phenotype); the ethylene response gene (EIN2) (reduced expression resulting in a growth stature phenotype); the LFY gene (reduced expression resulting in a flower development phenotype); and, the RCY1 gene (reduced expression resulting in a virus resistance phenotype).

FIG. 2 shows a schematic diagram of the FAD2TASwt construct. The chimeric polynucleotide was constructed such that the target site for Arabidopsis miRNA was used as trigger sequence and was operably linked to the 5′ end of a silencer sequence. The silencer sequence comprises a synthetic DNA fragment containing 5 repeated copies of a 21 nucleotide segments complementary to the Arabidopsis fatty acid desaturase 2 (FAD2) gene. The trigger and silencer sequence are flanked by sequences derived from the TAS1c5′ and 3′ regions/structural elements, respectively.

FIG. 3 shows plant lines expressing the construct with the correct trigger sequence to miR173 (the FAD2TASwt chimeric polynucleotide (SEQ ID NO: 15)) have increased levels of high oleic acid, as would be expected when FAD2 is silenced. This is not seen in the control plants (those designated with letters instead of numbers) where the trigger sequence is not homologous to miR173 (expressing SEQ ID NO: 16, referred to as referred to as FAD2TASmut), nor is it seen in an untransformed plant (wt=wild type).

FIG. 4 provides non-limiting schematic diagrams for chimeric polynucleotides that employ a trigger sequence to miR171 and a PDS silencer sequence.

FIG. 5 provides non-limiting schematic diagrams for chimeric polynucleotides that can be used to target suppression of Fad2.

FIG. 6 provides the TAS1a locus from Arabidopsis. The miRNA target sequence is underlined, and the known ta-siRNA sequences are double underlined. The polynucleotide sequence of TAS1a is set forth in SEQ ID NO:24.

FIG. 7 provides the TAS1b locus from Arabidopsis. The miRNA target sequence is underlined, and the known ta-siRNA sequences are double underlined. The polynucleotide sequence of TAS1b is set forth in SEQ ID NO:25.

FIG. 8 provides the TAS1c locus from Arabidopsis. The miRNA target sequence is underlined and the known ta-siRNA sequences are double underlined. The polynucleotide sequence of TAS1c is set forth in SEQ ID NO:26.

FIG. 9 provides the TAS2 locus from Arabidopsis. The miRNA target sequence is underlined, and the known ta-siRNA sequences are double underlined. The polynucleotide sequence of TAS2 is set forth in SEQ ID NO:27.

FIG. 10 provides the TAS3 locus from Arabidopsis. The miRNA target sequence is underlined, and the known ta-siRNA sequences are double underlined. The polynucleotide sequence of TAS3 is set forth in SEQ ID NO: 19.

FIG. 11 provides the ZmTAS3 locus from Zea mays. The miRNA target sequence is underlined, and the known ta-siRNA sequences are double underlined. The polynucleotide sequence of ZmTAS3 is set forth in SEQ ID NO:17.

FIG. 12 provides the GmTAS3 locus from Soybean. The miRNA target sequence is underlined, and the known ta-siRNA sequences are double underlined. The polynucleotide sequence of GmTAS3 is set forth in SEQ ID NO:28.

### BRIEF SUMMARY OF THE INVENTION

Methods and compositions are provided for reducing the level of expression of a target polynucleotide of interest. The methods and compositions selectively silence the target polynucleotide of interest by linking in a chimeric polynucleotide construct the target for a sRNA to a sequence corresponding to all or part of the gene or genes to be silenced.

Compositions comprising a chimeric polynucleotide comprising a trigger sequence operably linked to a silencer sequence of an endogenous or a native target polynucleotide are provided. The silencer sequence can be orientated in the chimeric polynucleotide to produce a sense or an anti-sense transcript of the target polynucleotide. The trigger sequence comprises a target for a miRNA or a siRNA.

In further compositions, the chimeric polynucleotide comprising the trigger sequence operably linked to the silencer sequence further comprises a nucleotide sequence comprising a sRNA that corresponds to the trigger sequence employed in the chimeric construct. In other compositions, the target polynucleotide is a polynucleotide from a second organism, such as a plant pest, that feeds upon the organism containing the chimeric polynucleotide(s).

In further compositions, the chimeric polynucleotide comprises at least one structural element of a trans-acting siRNA (TAS) encoding locus or a biologically active variant or fragment thereof. In such embodiments, at least one of the TAS ta-siRNA sequences is replaced with a heterologous silencing element. In other embodiments, a TAS ta-siRNA sequence is replaced with a heterologous silencing element and the TAS miRNA target site is replaced with a heterologous trigger sequence. Further provided are novel TAS encoding loci and biologically active variants and fragments thereof.

Compositions further comprise vectors, seeds, grain, cells, and organisms, including plants and plant cells, comprising the chimeric polynucleotide of the invention.

Methods are provided for reducing the level of expression of a target polynucleotide of interest. The method comprises introducing into a cell a chimeric polynucleotide comprising a trigger sequence operably linked to a silencer sequence of an endogenous target polynucleotide and expressing the chimeric polynucleotide in the cell. In specific methods, the trigger sequence is a target of a miRNA or a siRNA. In other methods, the target polynucleotide is a polynucleotide from a second organism, such as a plant pest, that feeds upon the organism containing the chimeric polynucleotide(s).

In further methods, the reduction in the expression level of the target polynucleotide in a plant or plant cell modulates fatty acid composition, such as, increasing the level of oleic acid in the seed of the plant. In still other methods, the reduction in the level of expression of the target polynucleotide modulates the level of at least one seed storage protein, so altering the nutritional value of the protein of the seed or the functionality of protein extract of the seed. Additional methods and compositions for modulating other agronomic traits are also provided including, but not limited to, modulations in flowering time, stalk strength, starch extractability, grain digestibility/energy availability, and/or reduced raffinoses.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention provides methods and compositions useful for silencing targeted sequences. The compositions can be employed in any type of plant cell, and in other cells which comprise the appropriate processing components (e.g., RNA interference components), including invertebrate and vertebrate animal cells. The methods can be adapted to work in any eukaryotic cell system. Additionally, the compositions and methods described herein can be used in individual cells, cells or tissue in culture, or in vivo in organisms, or in organs or other portions of organisms. In specific embodiments, the organism is non-human. Finally, the methods can be adapted to silence genes of a second organism that feeds or is a pest on the organism in which the compositions are expressed.

The compositions selectively silence the target polynucleotide by linking in a chimeric construct the target for a miRNA or siRNA to a sequence corresponding to all or part of the gene or genes to be silenced. Such miRNA or siRNAs will be collectively referred to as sRNAs (small RNAs). The target sequence for the sRNA when linked to the sequences corresponding to the gene or genes to be silenced will be referred to as the “trigger sequence.” The sequence corresponding to the gene or genes to be silenced will be referred to as the “silencer sequence.” The invention thus provides compositions comprising a chimeric polynucleotide comprising a trigger sequence operably linked to at least one silencer sequence. In specific embodiments, the chimeric polynucleotide can comprise appropriate regulatory elements. There are several ways to do this, which are outlined here; the person skilled in the art will observe that different combinations of the methods outlined here will be possible.

A chimeric polynucleotide comprising the target of a sRNA normally present in the cell or the organism as the trigger sequence operably linked to at least one silencer sequence comprising one or more sequences at least 19 nt long each corresponding to or complementary to one or more genes to be silenced in the organism of interest is transformed into that cell or organism. The trigger sequence must be at least long enough for the sRNA to effectively and specifically hybridize with the trigger. However, the trigger sequence can comprise sequences beyond the region complementary to the sRNA. Accordingly, the trigger sequence may be at least 15, 16, 17, 18, 19, 20, 21, 22, 23, nucleotides in length or up to the full-length complement of the corresponding sRNA, so long as the trigger sequence, when operably linked to the silencer sequence, is capable of reducing the level of expression of the target polynucleotide. The portion of the trigger sequence complementary to the sRNA must have sufficient complementarity with the sRNA, such as 78%, 80%, 85%, 90%, 91%, 92%, 93%, 94%, 95%, 96%, 97%, 98%, 99% or 100% sequence complementarity, to allow the trigger sequence, when operably linked to the silencer sequence, to reduce the level of expression of the target polynucleotide. In one embodiment, the portion of the trigger sequence complementary to the sRNA comprises no more than two consecutive mismatches to the sRNA, and no more than 4 mismatches in total. If the trigger sequence includes extraneous sequences beyond the region complementary to the sRNA, these extraneous sequences need have no homology to the sRNA.

In addition, the trigger sequence may be located either 5′, 3′, or internal to the silencer sequence or if multiple silencer sequences are employed in the construct, it can be located between such sequences. More than one copy of the trigger sequence may be included, with the different copies at different positions relative to the silencer sequence. Furthermore, two different trigger sequences could be used in the same chimeric construct, for example to trigger silencing in different cell types. The sRNA target is chosen on the basis of the natural presence of the sRNA in the cells or tissues of the organism to be transformed. Therefore, if it is desired to silence a gene at all times and in all parts of the organism, a sRNA target corresponding to a sRNA present at all times and in all parts of the organism would be chosen as the trigger sequence. Alternatively, if it is desired to silence the gene only in a particular tissue or development stage of the organism, a sRNA target corresponding to a sRNA present predominately in those tissues or developmental stages would be chosen. For example, if it were desired to silence a gene in the seeds of plants, one would choose as a trigger sequence the target sequence of a sRNA present only in the seeds. There are now numerous databases listing miRNAs or siRNAs present in different organisms and in different tissues, organs, or developmental stages of those organisms.

Alternatively, if a sRNA with the desired expression pattern is not available or known in the organism to be transformed, one can supply the sRNA in a separate polynucleotide construct or in the same chimeric construct. One would then use as the trigger sequence the target of the sRNA so used. If a miRNA target is used as a trigger sequence, the corresponding miRNA could be delivered by expressing the primary miRNA form (pri-miRNA) or the pre-miRNA form. siRNAs complementary to the trigger sequence could be provided in chimeric constructs in any number of forms, such as those described by Helliwell et al. (2005) Methods Enzymol 392:24-35, Wesley et al. (2004) Methods Mol Biol 265:117-29; and Helliwell et al. (2003) Methods 30:289-295, each of which is herein incorporated by reference, and similar methods known in the art for generating sRNAs. In other embodiments, a naturally occurring trans acting siRNA locus such as those described by Allen et al. ((2005) Cell 121:207-221) could be modified to include the siRNA corresponding to the trigger sequence. The sRNA used could be derived from the organism of interest or from another organism, and can be operably linked to a promoter that provides the desired expression pattern.

In both of the above embodiments, certain considerations apply to the silencer sequence; i.e., the sequences of the genes to be silenced included in the chimeric construct along with the trigger sequence. In principle, the silencer sequence may be as short as 19 bp each (Allen et al. (2005) Cell 121:207-221; Schwab et al. (2005) Developmental Cell 8:517-527). In other embodiments, the silencer sequence may be at least about 20, 25, 30, 40, 50, 60, 70, 80, 90, 100, 120, 140, 160, 180, 200, 250, 300, 350, 400, 450, 500, 550, 600, 650, 700, 750, or up to the full-length of the targeted transcript. In specific embodiments, the silencer sequence will be between about 100 and 300 nt. In addition, the silencer sequence may represent either strand of the gene to be silenced. Accordingly, the silencer sequence can have at least 75%, 80%, 85%, 90%, 91%, 92%, 93%, 94%, 95%, 96%, 97%, 98%, 99%, or 100% sequence identity or sequence complementarity to the transcript of the target polynucleotide. The silencer sequence may be derived from various sequences, including but not limited to, the coding sequence of the gene to be silenced, the 5′ untranslated region, the 3′ untranslated region, the promoter of the gene to be silenced, or any combination thereof.

The trigger sequence can be contiguous or non-contiguous with the operably linked silencer sequence. A non-contiguous, operably linked trigger sequence and silencer sequence can be about 1 to about 5, about 5 to about 10, about 10 to about 20, about 20 to about 30, about 30 to about 40, about 40 to about 50, about 50 to about 100, about 100 to about 200, about 200 to about 500, about 500 to about 1000, about 1000 to about 2000 nucleotides apart or any integer or more nucleotides apart.

The gene to be silenced need not be present in the organism to be transformed. Various workers (U.S. Application Publication No. 20040187170; 20040133943; 20040068761; 20030051263; U.S. Pat. No. 6,506,559; and, WO2005/019408, each of which is herein incorporated by reference) have shown that pests or pathogens of an organism may be defended against by the expression of double stranded RNAs corresponding to genes required for the viability or reproduction of the pest in the organism to be protected in such a way that these are taken up by the pest. The methods and compositions of the present invention, in all their embodiments, provide an alternative technique to provide such double stranded RNA. Specifically, the trigger sequence can be operably linked to at least one silencer sequence corresponding to a gene or fragment of a gene required for the viability or reproduction of the pest. In specific embodiments, the chimeric polynucleotide includes a promoter in cells or tissues attacked by the pest or pathogen. Again, the trigger sequence could correspond to a sRNA normally present in such cells, or a suitable sRNA corresponding to the trigger could be provided in a construct driven by a similar promoter delivered in the same or in a parallel polynucleotide construct. For example, plant pests that could be combated in this way include insects, nematodes, and fungi.

In another embodiment, one can design constructs based on the trans-acting siRNA (TAS) encoding loci or biologically active variants or fragments thereof such as those described, for example, by Allen et al (2005) Cell 121:207-221 and Williams et al (2005) PNAS 102: 9703-9708. A TAS encoding locus comprises one or more ta-siRNA sequences, a miRNA target site and additional sequences which flank these elements which are referred to herein as “TAS structural elements.” Constructs of the invention that employ TAS encoding locus or biologically active variants or fragments thereof comprise a TAS encoding locus or a biologically active variant or fragment thereof wherein at least one of the TAS ta-siRNA sequences is replaced with a heterologous silencer sequence. In other embodiments, at least one of the TAS ta-siRNA sequences is replaced with a heterologous silencer sequence and at least one of the TAS miRNA target sites is replaced with at least one heterologous trigger sequence. The expression of the chimeric polynucleotide in a cell reduces the level of expression the endogenous target polynucleotide.

As used herein, the term “structural element of a TAS encoding locus” comprises any fragment of a TAS encoding loci (i.e., a fragment comprising at least 20, 30, 50, 70, 90, 110, 130, 150, 170, 190, 210, 230, 250, 270, 290, or more polynucleotides). Alternatively, the structural element of a TAS encoding locus can share at least 80%, 85%, 90%, 91%, 92%, 93%, 94%, 95%, 96%, 97%, 98%, 99% or greater sequence identity across the full length of the TAS encoding locus or across a fragment or domain thereof. Such a “structural element of a TAS encoding locus”, when operably linked to a silencing sequence and a trigger sequence and expressed in a cell, reduces the level of a target polynucleotide.

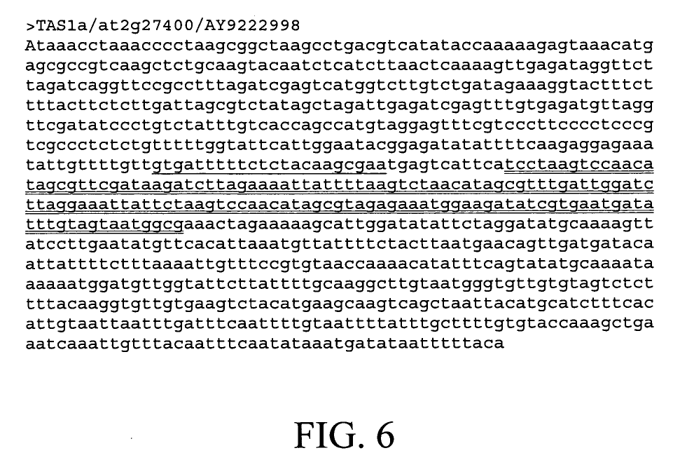

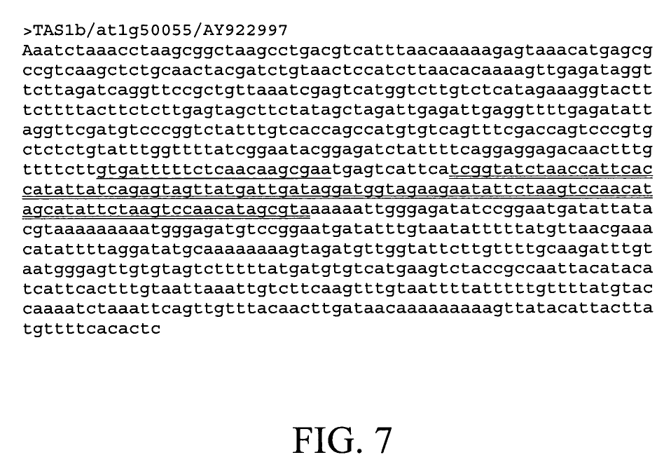

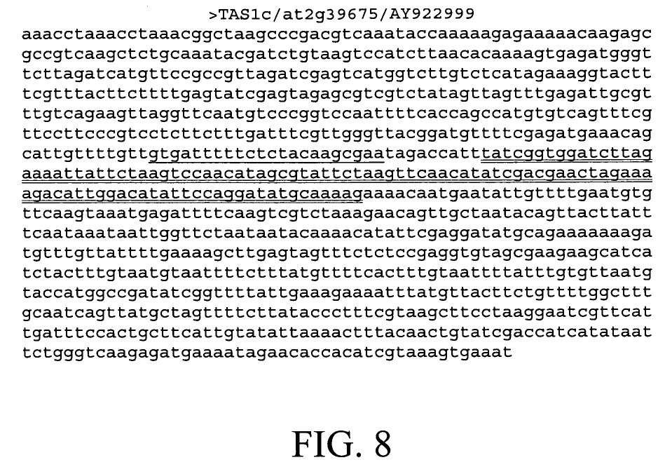

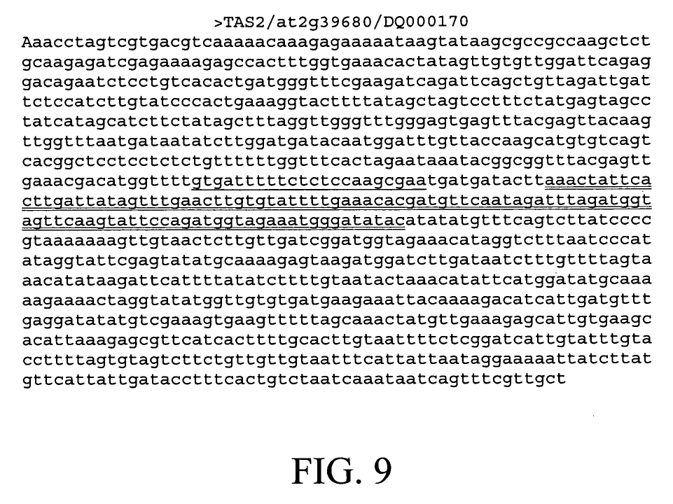

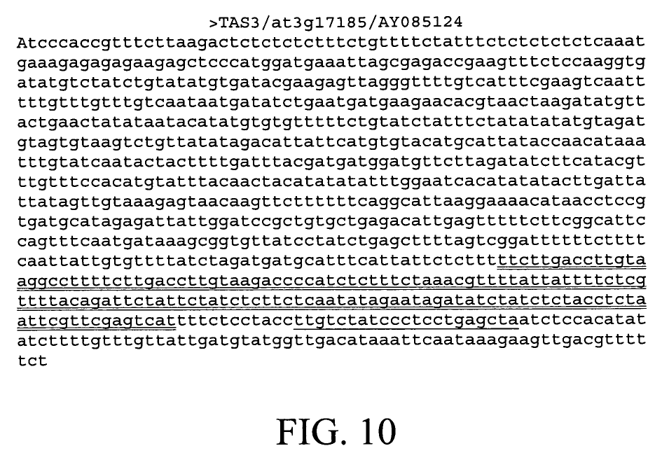

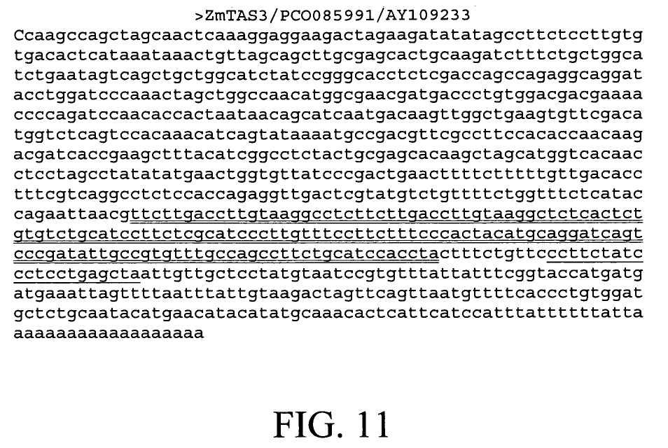

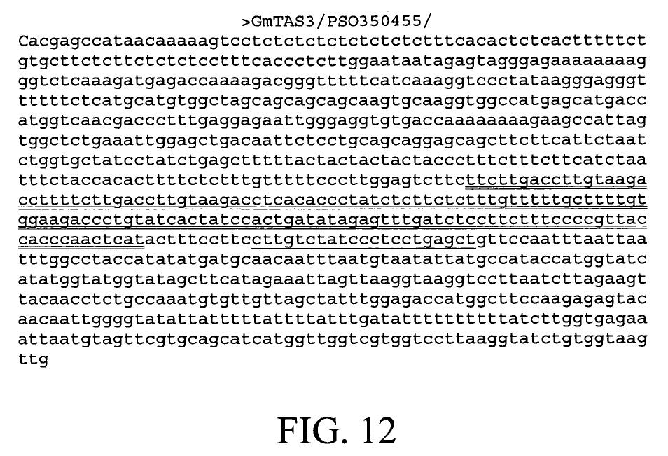

Non-limiting examples of TAS loci are set forth in SEQ ID NOS: 24-28, 17 and 19. FIGS. 6-12 further denote the ta-siRNA sequences and the miRNA targets sites in these non-limiting TAS encoding loci. For example, the TAS1c locus contains at least 205 nucleotides 5′ of the target site for the miRNA. Other TAS loci such as TAS3, homologues of which are found in Arabidopsis, soybean and maize, have flanking sequences 3′ of the miRNA target site, which unlike the miRNA target site in TAS1c sets a register that runs in reverse (i.e., the ta-siRNA from the TAS3 locus are derived from sequences 5′ to the miRNA target site).

The chimeric polynucleotides of the invention that employ TAS encoding loci or biologically active variants or fragments thereof, are based on the same principles as described earlier (a trigger sequence linked to a silencer sequence) but adding TAS structural elements. Thus, for example, one can replace the ta-siRNA encoding sequences of the TAS1c locus with one or more than one (2, 3, 4, 5 or more) silencer sequences, including 21 mers targeting the FAD2, APETALA1, or even both in the same construct. Such a chimeric construct could be operably linked to the 35S promoter, transformed into and expressed in a plant of interest (such as Arabidopsis), and the plants screened for high oleic oil, apetala1⁻ floral mutants, or both. One could also replace the miRNA target site of TAS1c, replacing the miR173 recognition site with any trigger sequence, including for example, that of miR167 and again screening for high oleic oil or apetala1⁻ phenotype depending on which silencer sequences were incorporated. The miRNA target site could also be replaced by that of a miRNA supplied in a separate chimeric construct under the control of a promoter of any desired specificity. The flanking regions of TAS1c or biologically active variants or fragments thereof would be maintained in such constructs. Of course this concept is not limited to TAS1. As noted above, TAS3 has a slightly different structure than TAS1. In the case of TAS3, ta-siRNA are derived from the 5′ cleavage fragment formed after miR390 binds to its target site on the locus causing cleavage. One could make a construct where a promoter, such as 35S, is operably linked to a modified TAS3-encoding chimeric gene. In place of endogenous ta-siRNA sequences, 21 nucleotide fragments homologous to FAD2, or any other desired target for gene silencing, could be incorporated. The construct would then be transformed into a plant of interest (such as Arabidopsis) and, in the case of a FAD2 target, the resulting plants could be assayed for high oleic acid content. Since there is a TAS3 homolog in maize (ZmTAS3), one could make a construct where a maize promoter, such as that for the maize ubiquitin gene, is operably linked to a modified ZmTAS3 encoding gene. In place of endogenous ta-siRNA sequences, 21 base sequences homologous to PDS, or any other desired target for gene silencing, could be incorporated. The construct would then be transformed into maize and in the case of a PDS target, the resulting plants could be assayed for photo-bleaching phenotype. A soybean homologue (GmTAS3: SEQ ID NO:28) is also provided. Accordingly, one could use the SCP1 promoter (Lu et al. (2000) Proc 15^{th} Internatl Sunflower Conference, June 2000, Toulouse, France, Abstr No K72-77 and U.S. Pat. No. 6,555,673) operably linked to a modified GmTAS3 encoding locus. In place of endogenous ta-siRNA sequences, 21 base sequences homologous to FAD2, for example, could be incorporated. The construct would then be transformed into soybean or soybean embryos and the resulting plants or embryos could be assayed for high oleic acid content. In other embodiments, in all these cases rather than targeting just one gene for silencing, multiple genes can be targeted by including silencers targeting multiple genes in one chimeric construct.

Other variations are conceivable and form other embodiments of this invention. Rather than using 21 mers in the TAS-derived structure, one could make a construct where a promoter, such as 355, is operably linked to a modified TAS1c encoding gene. In place of the endogenous ta-siRNA sequences, a longer fragment of FAD2, 25 or 50 or 100 or 150 or 200 or 250 or more nucleotides, could be incorporated. Again, the flanking sequences of TAS1c are left in place. The construct would then be transformed into Arabidopsis and the resulting plants could be assayed for high oleic acid content. One could target other genes, or target multiple genes by including fragments of more than one gene in the place of the endogenous ta-siRNA sequences.

Other embodiments, all based on those above, including but not limited to plants, cells, and seeds comprising the chimeric polynucleotide(s), are provided. Typically, the cell will be a cell from a plant, but other cells are also contemplated, including but not limited to fungal, insect, nematode, or animal cells. Plant cells include cells from monocots and dicots.

sRNAs which could be used to implement the present invention are well described, both in terms of sequence and function and expression pattern. For example, miR172 has been found to regulate flowering time and floral organ identity in Arabidopsis (Aukerman and Sakai (2003) Plant Cell 15: 2730-2741; Chen (2004) Science 303: 2022-2025). Also in Arabidopsis, miR319 and miR164 have been found to regulate leaf and root development, respectively (Palatnik et al. (2003) Nature 425: 257-263; Guo et al. (2005), Plant Cell 17:1376-1386). In maize, miR166 has been found to regulate leaf polarity (Juarez et al. (2004) Nature 428: 84-88). These represent only a very small number of the sRNAs of potential use; in fact the skilled artisan will find databases on the internet containing hundreds of sRNAs. For example, the miRNA Registry, run by the Sanger Institute, contains information on all known miRNAs in both plants and animals (Griffiths-Jones (2004) Nucleic Acids Research 32: D109-111; www.sanger.ac.uk/Software/Rfam/mirna/index.shtml). The Arabidopsis Small RNA Project contains information on cloned miRNAs and siRNAs in Arabidopsis (Gustafson et al. (2005) Nucleic Acids Research 33: 637-640; asrp.cgrb.oregonstate.edu/). A third database, MicroRNAdb, is also accessible online (166.111.30.65/micrornadb/). It can be expected that the range of sRNAs available will continue to grow.

The present invention further provides a novel TAS encoding loci set forth in SEQ ID NO:28. The sequence shares homology to TAS3 from both maize and Arabidopsis. Accordingly, the present invention provides for an isolated polynucleotide selected from the group consisting of (a) the polynucleotide set forth in SEQ ID NO: 28; (b) the polynucleotide having at least 80%, 85%, 90%, 91%, 92%, 93%, 94%, 95%, 96%, 97%, 98%, or 99% sequence identity to the sequence set forth in SEQ ID NO:29, wherein said polynucleotide retains the ability to reduce the level of a target polynucleotide; and, (c) the polynucleotide having at least 50, 100, 150, 200, 250, 300, 350, consecutive nucleotides of SEQ ID NO:28 or up to the full length of SEQ ID NO:28, wherein said polynucleotide retains the ability to reduce the level of a target polynucleotide. Plants, plant cells, seeds, and grain having a heterologous copy of the TAS3 locus set forth in SEQ ID NO:28 or a biologically active variant or fragment thereof are also provided.

Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Numeric ranges recited within the specification are inclusive of the numbers defining the range and include each integer within the defined range. Amino acids may be referred to herein by either commonly known three-letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

In the context of this disclosure, a number of terms shall be utilized. The terms “polynucleotide” and “nucleic acid” are used interchangeably herein. These terms encompass nucleotide sequences and the like. A polynucleotide may be a polymer of RNA or DNA that is single- or double-stranded and can contain natural, synthetic, non-natural and/or altered nucleotide bases. A polynucleotide in the form of a polymer of DNA may be comprised of one or more segments of cDNA, genomic DNA, synthetic DNA, or mixtures thereof.

The term “isolated” polynucleotide is one that (1) has been substantially separated or purified from other polynucleotides of the organism in which the polynucleotide naturally occurs, i.e., other chromosomal and extrachromosomal DNA and RNA, by conventional nucleic acid purification methods or (2) if the material is in its natural environment, the material has been altered by deliberate human intervention to a composition and/or placed at a locus in the cell other than the locus native to the material. The term also embraces recombinant polynucleotides and chemically synthesized polynucleotides.

As used herein, “substantially similar” and “substantially identical” are synonymous and refer to polynucleotides having nucleic acid sequences wherein changes in one or more nucleotide base result in substitution, deletion, and/or addition of one or more amino acids that do not affect the functional properties of the polypeptide encoded by the nucleic acid sequence. “Substantially identical” also refers to polynucleotides wherein changes in one or more nucleotide base do not affect the ability of the nucleic acid sequence to mediate alteration of gene expression by antisense or co-suppression technology among others. “Substantially identical” also refers to modifications of the nucleic acid fragments or polynucleotides (including a silencer sequence and/or the trigger sequence) of the embodiments, such as deletion, substitution and/or insertion of one or more nucleotides that do not substantially affect the functional properties of the resulting transcript vis-à-vis the ability to mediate gene silencing. “Substantially identical” refers to polynucleotides which are about 99%, about 98%, about 97%, about 96%, about 95%, about 94%, about 93%, about 92%, about 91%, about 90%, about 85%, about 80%, about 75%, or about 70% identical. Thus, a biologically active variant of a trigger sequence or a silencing sequence may differ from the native sequence (or the complement thereof) by between 1 and 30 nucleotides, or about 25, 20, 25, 10, 9, 8, 7, 6, 5, 4, 3, 2, or 1 nucleotide residues. The percentage of identity may be calculated with any of the programs described herein below, for instance, they may be calculated with the program GAP as described herein below. It is therefore understood that the embodiments of the invention encompass more than the specific exemplary sequences.

Moreover, substantially identical polynucleotides may also be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridisation, IRL Press, Oxford, U.K.). Stringency conditions can be adjusted to screen for moderately similar polynucleotides, such as homologous sequences from distantly related organisms, to highly similar polynucleotides, such as genes that duplicate functional enzymes from closely related organisms. Post-hybridization washes determine stringency conditions. One set of preferred conditions uses a series of washes starting with 6×SSC, 0.5% SDS at room temperature for 15 minutes, then repeated with 2×SSC, 0.5% SDS at 45° C. for 30 minutes, and then repeated twice with 0.2×SSC, 0.5% SDS at 50° C. for 30 minutes. A more preferred set of stringent conditions uses higher temperatures in which the washes are identical to those above except for the temperature of the final two 30 minute washes in 0.2×SSC, 0.5% SDS was increased to 60° C. Another preferred set of highly stringent conditions uses two final washes in 0.1×SSC, 0.1% SDS at 65° C.

Methods of alignment of sequences for comparison are well known in the art. Thus, the determination of percent sequence identity between any two sequences may be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 87:2264-2268, modified as in Karlin and Altschul (1993) Proc. Natl. Acad. Sci. USA 90:5873-5877.

Computer implementations of these mathematical algorithms may be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs may be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:0881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See, for example, the world wide web site for NCBI at ncbi.nlm.nih.gov (accessed by entering this address into a web browser, preceded by the “www.” prefix). Alignment may also be performed manually by inspection.

Unless otherwise stated, nucleotide sequence identity/similarity values provided herein refer to the value obtained using GAP Version 10 using the following parameters: % identity and % similarity for a nucleotide sequence using Gap Weight of 50 and Length Weight of 3, and the nwsgapdna.cmp scoring matrix. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used for peptide alignments in Version 10 of the GCG Wisconsin Genetics Software Package is BLOSUM62 (see Henikoff and Henikoff (1989) Proc. Natl. Acad. Sci. USA 89:10915).

As used herein, “sequence identity” or “identity” in the context of two polynucleotides makes reference to the residues in the two sequences that are the same when aligned for maximum correspondence over a specified comparison window. A “complement sequence” in the context of two oppositely orientated polynucleotides make reference to the nucleotide residues which when aligned interact to form a double-stranded structure (i.e., the complementary sequence to 5′-G-T-A-C-3′ is 3′-C-A-T-G-5′).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity. As used herein “percent complementarity” means the value determined by comparing the complementarity of two oppositely orientated polynucleotides. The percentage is calculated by determining the number of positions at which the complement nucleic acid base occurs in both sequences to yield the number of complement positions, dividing the number of complement positions by the total number of positions in the window of comparison, and multiplying the result by 100 to yield the percentage of sequence complementarity.

“Synthetic polynucleotide fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized,” as related to polynucleotide fragments, means that the component nucleotides were assembled in vitro. Manual chemical synthesis of polynucleotide fragments may be accomplished using well-established procedures, or automated chemical synthesis can be performed using one of a number of commercially available machines.

“Coding sequence” refers to a nucleotide sequence that encodes a specific protein (amino acid sequence), structural RNA, microRNA or siRNA. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences. “Gene” refers to a combination of a polynucleotide and the necessary regulatory sequences to direct the expression of the product of the gene. “Endogenous” gene or polynucleotide refers to a gene or polynucleotide present in a cell and expressed in trans to the chimeric polynucleotide of the invention. The endogenous gene can be native to the cell or heterologous to the host cell. A “native” polynucleotide or gene refers to a gene or a polynucleotide as found in nature, in either its natural location in the genome or in a different location in the genome. As used herein, “heterologous” in reference to a polynucleotide is a nucleic acid that originates from a foreign species, or is synthetically designed, or, if from the same species, is substantially modified from its native form in composition and/or genomic locus by deliberate human intervention.

As used herein, a chimeric polynucleotide comprises at least two elements which are heterologous with respect to one another. For example, a chimeric polynucleotide can comprise a coding sequence operably linked to a transcription initiation region that is heterologous to the coding sequence. Accordingly, a chimeric polynucleotide may comprise regulatory sequences, silencer sequences, trigger sequences, and/or coding sequences that are derived from different sources, or regulatory sequences, coding sequences, silencer sequences and/or trigger sequences derived from the same source, but arranged in a manner different than that found in nature. In another example, a silencer sequence is heterologous to a trigger sequence if such elements are normally not present in the same polynucleotide (i.e., transcript) or the elements are present in the same polynucleotide but have been modified from their native form in composition or their position within the polynucleotide (i.e., transcript). A chimeric polynucleotide may also comprise sequences encoding RNAs that take a form that might or might not be found in nature, such as chimeric polynucleotides designed to produce dsRNAs that will be converted to siRNAs or miRNAs.

A “foreign” polynucleotide refers to a gene not normally found in the host organism, but that is introduced into the host organism by gene transfer. Foreign genes may comprise native polynucleotides inserted into a non-native organism, a heterologous polynucleotide, or a chimeric polynucleotide. A “transgene” is a polynucleotide that has been introduced into a cell by a transformation procedure.

“Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. When used to refer to the joining of a silencer sequence and a trigger sequence, by operably linked is intended that these two elements are joined such that their transcript has the ability to reduce the level of expression of the target polynucleotide. Polynucleotides may be operably linked to regulatory sequences in sense or antisense orientation.

The term “recombinant polynucleotide construct” means, for example, that a recombinant polynucleotide is made by an artificial combination of two otherwise separated nucleotide segments, e.g., by chemical synthesis or by the manipulation of isolated segments of nucleic acids by genetic engineering techniques.

The term “introduced” means providing a polynucleotide or protein into a cell. Introduced includes reference to the incorporation of a polynucleotide into a eukaryotic or prokaryotic cell where the polynucleotide may be incorporated into the genome of the cell, and includes reference to the transient provision of a polynucleotide or protein to the cell. Introduced includes reference to stable or transient transformation methods, as well as sexually crossing.

“Promoter” refers to a polynucleotide capable of controlling the expression of a polynucleotide. In general, the polynucleotide to be transcribed is located 3′ to a promoter sequence. The promoter sequence may comprise proximal and more distal upstream elements; the latter elements often referred to as enhancers. Accordingly, an “enhancer” is a polynucleotide, which can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions. New promoters of various types useful in plant cells are constantly being discovered; numerous examples may be found in the compilation by Okamuro and Goldberg ((1989) Biochem. Plants 15:1-82; see also Potenza et al. (2004) In Vitro Cell. Dev. Biol.—Plant 40: 1-22). It is further recognized that since in most cases the exact boundaries of regulatory sequences have not been completely defined, polynucleotide fragments of different lengths may have identical promoter activity.

A number of promoters can be used, these promoters can be selected based on the desired outcome. It is recognized that different applications will be enhanced by the use of different promoters in plant expression cassettes to modulate the timing, location and/or level of expression of the miRNA. Such plant expression cassettes may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Constitutive, tissue-preferred or inducible promoters can be employed. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. If low level expression is desired, weak promoter(s) may be used. Weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

Examples of inducible promoters are the Adh1 promoter, which is inducible by hypoxia or cold stress, the Hsp70 promoter, which is inducible by heat stress, the PPDK promoter and the pepcarboxylase promoter, which are both inducible by light. Also useful are promoters which are chemically inducible, such as the In2-2 promoter which is safener induced (U.S. Pat. No. 5,364,780), the ERE promoter which is estrogen induced, and the Axig1 promoter which is auxin induced and tapetum specific but also active in callus (PCT US01/22169).

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter, Boronat et al. (1986) Plant Sci. 47:95-102; Reina et al. Nucl. Acids Res. 18(21):6426; and Kloesgen et al. (1986) Mol. Gen. Genet. 203:237-244. Promoters that express in the embryo, pericarp, and endosperm are disclosed in U.S. Pat. No. 6,225,529 and PCT publication WO 00/12733. The disclosures each of these are incorporated herein by reference in their entirety.

In some embodiments it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Promoters that are expressed locally at or near the site of pathogen infection can also be used. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant. Path. 41:189-200).

Additionally, as pathogens find entry into plants through wounds or insect damage, a wound-inducible promoter may be used in the constructions of the polynucleotides. Such wound-inducible promoters include potato proteinase inhibitor (pin II) gene (Ryan (1990) Ann. Rev. Phytopath. 28:425-449; Duan et al. (1996) Nature Biotech. 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIPI (Rohmeier et al. (1993) Plant Mol. Biol. 22:783-792; Eckelkamp et al. (1993) FEBS Lett. 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of a sequence of interest within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590. In addition, the promoters of cab and ribisco can also be used. See, for example, Simpson et al. (1958) EMBO J 4:2723-2729 and Timko et al. (1988) Nature 318:57-58.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691. See also U.S. Pat. Nos. 5,837,876; 5,750,386; 5,633,363; 5,459,252; 5,401,836; 5,110,732; and 5,023,179. The promoter from the phaseolin gene could also be used (Murai et al. (1983) Science 23:476-482 and Sengopta-Gopalen et al. (1988) PNAS 82:3320-3324).

The “3′non-coding region” or “terminator region” refers to DNA or RNA sequences located downstream of a coding sequence and may include polyadenylation recognition sequences and other sequences encoding regulatory signals capable of affecting mRNA processing or gene expression. The polyadenylation signal is usually characterized by effecting the addition of polyadenylic acid tracts to the 3′ end of the mRNA precursor. The use of different 3′ non-coding sequences is exemplified by Ingelbrecht et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and that may be translated into protein by the cell. “cDNA” refers to a DNA that is complementary to and derived from an mRNA. The cDNA may be single-stranded or converted into the double stranded form using, for example, the Klenow fragment of DNA polymerase I. “Functional RNA” refers to sense RNA, antisense RNA, ribozyme RNA, transfer RNA, miRNA, siRNA or other RNA that may not be translated but yet has an effect on cellular processes.

The term “plant” as used herein encompasses a plant cell, plant tissue (including callus), plant part, plant cells that are intact in plant or parts thereof, whole plant, ancestors and progeny. A plant part may be any part or organ of the plant and include for example a seed, fruit, stem, leaf, shoot, flower, anther, root or tuber. The term “plant” also encompasses suspension cultures, embryos, meristematic regions, callus tissue, gametophytes, sporophytes, pollen, and microspores. The plant as used herein refers to all plants including algae, ferns and trees. Grain is intended to mean the mature seed produced by commercial growers for purposes other than growing or reproducing the species. In a preferred embodiment the plant belongs to the superfamily of Viridiplantae, further preferably is a monocot or a dicot. Specific reference is made to the more than 700 host plants described in Sasser (1980) Plant Disease 64:36-41) including most cultivated crops, ornamentals, vegetables, cereals, pasture, trees and shrubs.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), banana (Musa acuminata and Musay x paradisiaca), vine, pear (Pyrus communis), apple, rapeseed, oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are employed, and in yet other embodiments corn plants are employed.

The term “expression” or “expressing,” as used herein refers to the transcription of a polynucleotide. Expression may also refer to the translation of mRNA into a polypeptide. “Overexpression” refers to the production of a gene product in an organism that exceeds the level of production in a control organism.

The term “silencing” refers collectively to a variety of techniques used to suppress or turn off expression of a gene, so that the product of the gene is not present or present at a reduced level in an organisms that is below the level found in a control organism. As used herein, reduced level means decreased, reduced, lowered, prevented, inhibited, stopped, suppressed, eliminated, and the like. Reduced level includes expression that is decreased by about 10%, 15%, 20%, 25%, 30%, 35%, 40%, 45%, 50%, 55%, 60%, 65%, 70%, 75%, 80%, 85%, 90%, 95%, or 100% relative to the appropriate control organism. A reduction in the expression of a polynucleotide of interest may occur during and/or subsequent to growth of the organism (i.e., plant) to the desired stage of development. As described earlier, “RNAi” refers to a series of related techniques to reduce the expression of genes (See for example U.S. Pat. No. 6,506,559).

A “subject organism or cell” is one in which genetic alteration, such as transformation, has been effected as to a gene of interest, or is an organism or cell which is descended from an organism or cell so altered and which comprises the alteration. A “control” or “control organism” or “control cell” provides a reference point for measuring changes in phenotype of the subject organism or cell.

A control organism or cell may comprise, for example: (a) a wild-type organism or cell, i.e., of the same genotype as the starting material for the genetic alteration which resulted in the subject organism or cell; (b) an organism or cell of the same genotype as the starting material but which has been transformed with a null construct (i.e. with a construct which has no known effect on the trait of interest, such as a construct comprising a marker gene or a construct having a non-functional trigger sequence and/or silencer sequence); (c) an organism which is a non-transformed segregant among progeny of a subject organism; and, (d) an organism or cell genetically identical to the subject organism or cell but which is not exposed to conditions or stimuli that would induce expression of the chimeric polynucleotide.

The reduced expression level of the target polynucleotide may be measured directly, for example, by assaying for the level of the target polynucleotide expressed in the cell or the organism, or, in specific embodiments, assaying for the level of the polypeptide encoded thereby. The reduced expression level of the target polynucleotide can also be assayed indirectly, for example, by measuring the activity of the target polynucleotide or, in specific embodiments, assaying for the activity of the polypeptide encoded thereby.

“Stable transformation” is intended to mean that the polynucleotide construct introduced into a cell integrates into the genome of the cell and is capable of being inherited by the progeny thereof. “Transient transformation” is intended to mean that a polynucleotide is introduced into the cell and does not integrate into the genome of the cell or a polypeptide is introduced into a cell.

Host organisms containing the introduced polynucleotide are referred to as “transgenic” organisms. By “host cell” is meant a cell that contains an introduced polynucleotide construct and supports the replication and/or expression of the construct. Host cells may be prokaryotic cells such as E. coli, or eukaryotic cells such as fungi, yeast, insect, amphibian, nematode, or mammalian cells. Alternatively, the host cells are monocotyledonous or dicotyledonous plant cells. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050), among others. In some embodiments, transient expression may be desired. In those cases, standard transient transformation techniques may be used. Such methods include, but are not limited to viral transformation methods, and microinjection of DNA or RNA, as well other methods well known in the art.

Standard recombinant DNA and molecular cloning techniques used herein are well known in the art and are described more fully in Sambrook et al. (1989) Molecular Cloning: A Laboratory Manual; Cold Spring Harbor Laboratory Press: Cold Spring Harbor, (hereinafter “Sambrook”). Plasmid vectors comprising the isolated polynucleotide of the invention may be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al. (1985) EMBO J. 4:2411-2418; De Almeida et al. (1989) Mol. Gen. Genetics 218:78-86), and thus that multiple events may have to be screened in order to obtain lines displaying the desired expression level and pattern. Such screening may be accomplished by PCR or Southern analysis of DNA to determine if the introduced polynucleotide is present in complete form, and then northern analysis or RT-PCR to determine if the expected RNA is indeed expressed.

“PCR” or “polymerase chain reaction” is a technique for the synthesis of large quantities of specific DNA segments. It consists of a series of repetitive cycles (Perkin Elmer Cetus Instruments, Norwalk, Conn.). Typically, the double-stranded DNA is heat denatured, the two primers complementary to the 3′ boundaries of the target segments are annealed at low temperature and then extended at an intermediate temperature. One set of these three consecutive steps is referred to as a cycle. RT-PCR is a variation of PCR in which PCR reactions are preceded by a reverse transcriptase reaction to convert RNA into DNA, thus allowing the use of PCR to monitor RNA as well as DNA.

The methods provided can be practiced in any organism in which a method of transformation is available, and for which there is at least some sequence information for the gene(s) to be silenced or for a region flanking the gene(s) to be silenced. As described earlier two or more genes could be silenced using one chimeric construct, but it is also understood that two or more sequences could be targeted by sequential transformation or co-transformation with one or more chimeric genes of the type described.

General categories of polynucleotides of interest include, for example, those genes involved in regulation or information, such as zinc fingers, transcription factors, homeotic genes, or cell cycle and cell death modulators, those involved in communication, such as kinases, and those involved in housekeeping, such as heat shock proteins.

Polynucleotides targeted for silencing further include coding regions and non-coding regions such as promoters, enhancers, terminators, introns and the like, which may be modified in order to alter the expression of a polynucleotide of interest.

The polynucleotide targeted for silencing may be an endogenous sequence, a native sequence, or may be a heterologous sequence, or a transgene. For example, the methods may be used to alter the regulation or expression of a transgene. In specific embodiments, the polynucleotide targeted for silencing is not GFP. In other embodiments, the polynucleotide targeted for silencing imparts an agronomical trait to the plant. The polynucleotide targeted for silencing may also be a sequence from a pest or a pathogen; for example, the target sequence may be from a plant pest such as a virus, a mold or fungus, an insect, or a nematode. A chimeric polynucleotide of the type described herein could be expressed in a plant which, upon infection or infestation, would target the pest or pathogen and confer some degree of resistance to the plant.

In plants, other categories of polynucleotides targeted for silencing include genes affecting agronomic traits, insect resistance, disease resistance, herbicide resistance, sterility, grain characteristics, and commercial products. Genes of interest also included those involved in oil, starch, carbohydrate, or nutrient metabolism as well as those affecting, for example, kernel size, sucrose loading, and the like. The quality of grain is reflected in traits such as levels and types of oils, saturated and unsaturated, quality and quantity of essential amino acids, and levels of cellulose. For example, genes of the phytic acid biosynthetic pathway could be suppressed to generate a high available phosphorous phenotype. See, for example, phytic acid biosynthetic enzymes including inositol polyphosphate kinase-2 polynucleotides, disclosed in WO 02/059324, inositol 1,3,4-trisphosphate 5/6-kinase polynucleotides, disclosed in WO 03/027243, and myo-inositol 1-phosphate synthase and other phytate biosynthetic polynucleotides, disclosed in WO 99/05298, all of which are herein incorporated by reference. Genes in the lignification pathway could be suppressed to enhance digestibility or energy availability. Genes affecting cell cycle or cell death could be suppressed to affect growth or stress response. Genes affecting DNA repair and/or recombination could be suppressed to increase genetic variability. Genes affecting flowering time, stalk strength, starch extractability, reducing raffinoses, as well as genes affecting fertility could be silenced. Genes that modulate the fatty acid composition of the seed or gene that modulate the level of storage proteins in a seed could be silenced. Any sequence targeted for silencing could be suppressed in order to evaluate or confirm its role in a particular trait or phenotype, or to dissect a molecular, regulatory, biochemical, or proteomic pathway or network.

### EXPERIMENTAL

The present invention is further defined in the following Examples, in which all parts and percentages are by weight and degrees are Celsius, unless otherwise stated. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims. The disclosure of each reference set forth herein is incorporated by reference in its entirety.

### Example 1

### Silencing Using Trigger Sequences in Arabidopsis with a Color Marker as Supplementary Indicator

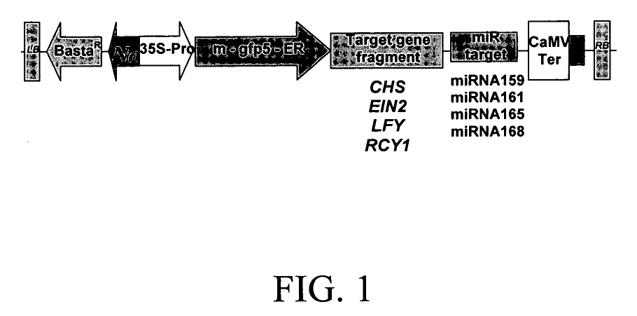

FIG. 1 shows the structure of the construct used. Between the left and right borders of a standard Agrobacterium transformation vector the following components are placed: the Basta selectable marker driven by the nos promoter and the 35S promoter driving a chimeric construct comprising the GFP polynucleotide, a silencer sequence (fragment of the gene to be silenced), and the trigger sequence followed by the terminators of the 35S gene. The four genes chosen as genes to be silenced are all involved in phenotypes such that loss of function is not lethal but is evident by simple visual inspection of the plants or virus inoculation. The four miRNA targets used as trigger sequences are chosen because the corresponding miRNAs are expressed in different tissues. miR159 is constitutive and abundant (SEQ ID NO:1); miR161 is not active in leaves (SEQ ID NO:2); miR165 (SEQ ID NO:3) and miR168 (SEQ ID NO:4 or 5) are active in leaves, but not as abundant as miR159. As controls, identical constructs are made in which the trigger sequence is mutated in such a way that the miRNA will no longer recognize the trigger.

Arabidopsis plants are transformed with each construct as described by (Clough and Bent (1998) Plant Journal 16:735-743). In each case, silencing is monitored by lack of fluorescence due to GFP and lack of the appropriate visual phenotype for each gene to be silenced: change in pigment content due to silencing of chalcone synthase, change in growth stature due to loss of expression of the ethylene response gene, change in floral morphology due to lack of leafy expression, and loss of viral resistance due to lack of rcy1 expression. RT-PCR and northern analysis are carried out to correlate these effects at a molecular level.

### Example 2

### Silencing Using Trigger Sequences Attached to Synthetic Arrays of 21 mers

A chimeric polynucleotide is constructed in which the target site for Arabidopsis miRNA (miR167; Reinhart et al. (2002) Genes and Development 16: 1616-1626) is used as trigger sequence and is operably linked to the 5′ end of a silencer sequence. The silencer sequence comprises a synthetic DNA fragment containing multiple 21 nucleotide segments complementary to the Arabidopsis fatty acid desaturase 2 (FAD2) gene. Each 21 nucleotide segment is designed to possess the characteristics required for efficient incorporation into RISC as described by Khvorova et al. ((2003) Cell 115: 199-208) and Schwarz et al. ((2003) Cell 115: 209-216). The 35S promoter and leader sequence (Odell (1985) Nature 313: 810-812) are attached to the 5′ end of the chimeric construct and the phaseolin transcriptional terminator (Barr et al. (2004) Molecular Breeding 13: 345-356) to the 3′ end. The entire chimeric polynucleotide is inserted into a standard binary vector and transformed into Arabidopsis. Transgenic plants containing the experimental construct are monitored for silencing of the FAD2 gene using fatty acid analysis (Browse et al. (1986) Analytical Biochemistry 152: 141-145) and compared to control plants. The latter are created in an identical way except that the trigger sequence is mutated to remove homology to miR167.

### Example 3

### Silencing Using Trigger Sequences in Soybean Embryos

In order to provide trigger sequences, miRNAs active in soybean embryos are cloned and characterized as follows: RNA is prepared from somatic embryos. The size fractionated sRNAs are ligated to 3′ and 5′ RNA-DNA adaptors, PCR amplified using adaptor-specific primers and cloned into plasmid vectors using standard procedures (Llave et al. (2002) Plant Cell 14, 1605-1619). Abundant sRNAs are identified from the sequence analysis of the cloned sRNAs and their complementary nucleotide sequence is incorporated as the trigger element of chimeric constructs as described below. Alternatively, constructs encoding exogenous miRNA can be expressed in the plant and the corresponding trigger sequence for the exogenous miRNA can be employed.

A. Silencing of a Lipid Biosynthetic Gene

i. A Chimeric Construct Comprising the Following is Constructed:

1. A silencer sequence comprising a 300 nt fragment from nucleotide 363 to nucleotide 662 of the open reading frame of the fatty acid desaturase 2 (FAD2) cDNA from soybean (U.S. Pat. No. 6,872,872 B1) is PCR-amplified from plasmid pSF2-169K (U.S. Pat. No. 6,872,872 B1) using primers designed to introduce NotI restriction enzyme sites at both ends of the fragment. The following primers, described in WO0200904 A2, are used:
5′-GAATTCGCGGCCGCCCAATCTATTGGGTTCTC-3′ (SEQ ID NO: 6)—primer position 363 in Fad2 sequence
GAATTCGCGGCCGCGAGTGTGACGAGAAGAGA-3′ (SEQ ID NO: 7)—primer position 662 in Fad2 sequence

The PCR products are cut with Not I and ligated into pBluescript and the sequence of the fragments is verified.

2. A trigger sequence complementary to one of the miRNAs is isolated in the steps outlined above. The miRNA-encoding fragment is PCR-amplified from one of the miR cDNAs described above using primers designed to introduce a BstEII site at both ends of the fragment. The PCR products are cut with BstEII and ligated into the Fad2-pBluescript vector described above. The sequence of the new fragment is verified.

The Not I digested FAD2-miRNA fragment is then ligated into the Not I site of plasmid pKR124 (described in WO2004071467 A2) which contains the promoter of the soybean Kunitz Trypsin Inhibitor gene (Jofuku et al. (1989) Plant Cell 1:1079-1093) and a hygromycin resistance gene cassette as a selectable marker. Silencing is monitored by examining the oleic acid content of individual embryos using gas chromatography as described in Example 7. Since lack of the enzyme encoded by the FAD2 gene disables conversion of oleic acid to linoleic acid, silencing can be monitored by assaying for high levels of oleic acid relative to control embryos. The latter are created using identical procedures and constructs, except that the trigger sequence will be altered to remove complementarity with the miRNA.

ii. The above experiment (Example 3.A.i) is repeated as described, except that as a trigger sequence a sequence complementary to Arabidopsis miRNA159 (UUUGGAUUGAAGGGAGCUCUA (SEQ ID NO: 1); for clarity, this is the sequence of miRNA159) is used, and the final construct in the soybean transformation vector is supplemented with a chimeric polynucleotide cassette comprising sequences encoding the precursor of Arabidopsis miRNA159 as previously described by Achard et al. ((2004) Development 131:3357-3365) cloned into soybean expression vector pJS92 (WO2004071467 A2 and WO2004071178 A2) such that the sequences encoding the precursor of Arabidopsis miRNA159 is operably linked to the soybean annexin promoter of pJS92. Transformation is carried out and silencing monitored as above. The control comprises embryos transformed with a vector lacking the Arabidopsis gene encoding the precursor of miRNA159.

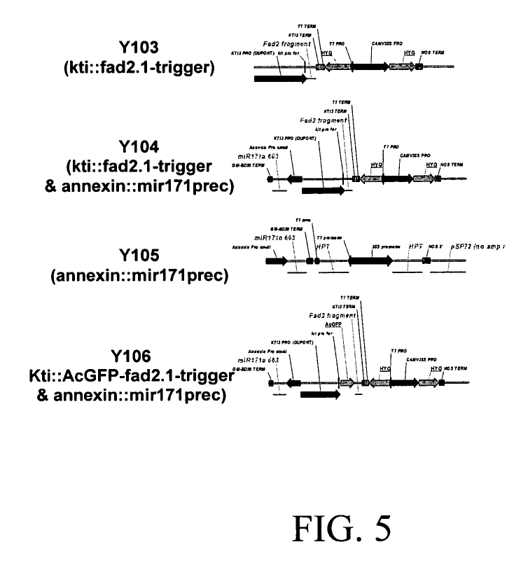

iii. The above experiment (example 3.A.i) is repeated as described, except that as a trigger sequence a sequence complementary to Arabidopsis miRNA171 (Bartel et al. (2003) Plant Physiology 132:709-717) is used, such that the trigger sequence is found 3′ of the silencer sequence. Non-limiting schematic diagrams of such silencing constructs are set forth in FIG. 5. In one embodiment, a polynucleotide encoding GFP is placed upstream of the silencer sequence. The final construct in the soybean transformation vector is supplemented with a chimeric polynucleotide cassette comprising sequences encoding the precursor of Arabidopsis miRNA171 as previously described by Bartel et al. (2003) Plant Physiology 132:709-717 cloned into soybean expression vector pJS92 (WO2004071467 A2 and WO2004071178 A2) such that the sequences encoding the precursor of Arabidopsis miRNA 171 is operably linked to the soybean annexin promoter of pJS92. Transformation is carried out and silencing monitored as above. The control comprises embryos transformed with a vector lacking the Arabidopsis gene encoding the precursor of miRNA 171.

B. Silencing of Seed Protein Genes

i. The three experiments above (example 3.A.i-3.A.iii) are repeated except that as silencing sequences, fragments of genes encoding soybean glycinin (GM-GY; a class of soybean seed storage proteins) are used. There are five genes in soybean encoding glycinins, which can be subdivided into two groups (Cho et al. (1989) Plant Cell 1:329-337). By using as a silencing sequence a recombinant GM-GY4/GY1-hybrid fragment, genes encoding both groups are silenced.

The recombinant DNA fragment GM-GY4/GY1-hybrid comprises a 634 polynucleotide fragment comprising 309 nucleotides from the soybean GM-GY4 gene and 325 nucleotides from the soybean GM-GY1 gene (Nielsen et al. (1989) Plant Cell 1:313-328) and is constructed by PCR amplification as follows:

1. An approximately 0.31 kb DNA fragment is obtained by PCR amplification using primers KS1 and KS2:

KS1: 5′-GCCAAGGAAAGCGTGAACAAGACCAG-3′ (SEQ ID NO: 8)

KS2: 5′-TGTGGCACGAACATTCATATTGGGCACTGA-3′ (SEQ ID NO: 9) using genomic DNA purified from leaves of Glycine max cv. Jack as a template.

2. An approximately 0.32 kb DNA fragment is obtained by PCR amplification using primers KS3 and KS4

KS3: 5′-TCAGTGCCCAATATGAATGTTCGTGCCACA-3′ (SEQ ID NO: 10)

KS4: 5′-GTTCTTTATCTGCCTGGCCTGCTGGC-3′ (SEQ ID NO: 11) also using genomic DNA purified from leaves of Glycine max cv. Jack as a template.

3. The 0.31 kb fragment and 0.32 kb fragment are gel purified using GeneClean (Qbiogene, Irvine Calif.), mixed and used as template for PCR amplification with KS1 and KS4 as primers to yield an approximately 634 bp fragment that is cloned into the commercially available plasmid pGEM-T Easy (Promega, Madison, Wis.).

4. The same triggers as used in silencing the lipid biosynthetic gene is used, but during PCR amplification primers designed to introduce a Spe I site at both ends of the fragment are used. The PCR products are cut with Spe I and ligated into the GM-GY4/GY1-hybrid in pGEM-T Easy vector described above. The sequence of the new fragment is verified.

5. The Not I digested GM-GY4/GY1-hybrid-miRNA fragment is then ligated into the Not I site of plasmid pKR124 (described in WO2004071467 A2) which contains the promoter of the soybean Kunitz Trypsin Inhibitor gene (Jofuku et al. (1989) Plant Cell 1:1079-1093) and a hygromycin resistance gene cassette as a selectable marker.

### Example 4

### Silencing Using Trigger Sequences in Corn Plants

A. A miRNA target for use as a trigger sequence is synthesized by designing a sequence that is the complement of a miRNA expressed in corn seedlings selected from the many Zea mays miRNAs described in the miRNA Registry, run by the Sanger Institute (Griffiths-Jones (2004) Nucleic Acids Research 32: D109-111; www.sanger.ac.uk/Software/Rfam/mirna/index.shtml) by typing “Zea mays” into the search window on the home page. The sequence is operably linked at the 3′ end to a 1361 nt fragment of the maize phytoene desaturase gene (PDS) (SEQ ID NO:12; Genbank accession number AAC12846, Li et al. (1992) J Hered 83:109-113). This combination is operably linked to the maize ubiquitin promoter (Christensen et al. (1989) Plant Mol. Biol. 12:619-632; Christensen et al. (1992) Plant Mol Biol 18:675-689). The resulting chimeric construct, comprising the ubiquitin promoter operably linked to a polynucleotide comprising a fragment of the PDS gene linked to the target of the chosen miRNA as trigger sequence is inserted into a standard vector for maize transformation and includes the bar gene as a selectable marker. The construct is transformed into maize using the procedure described in Example 6. The plants are regenerated to the plantlet stage. Silencing of the PDS gene is monitored by looking for white plantlets. Silencing of PDS interferes with carotenoid biosynthesis and results in bleaching of green tissue under high light conditions. As a control a completely parallel experiment is carried out exactly as described above except that the trigger sequence is altered such that complementarity to the miRNA is reduced.

In a variation of the above procedure, the trigger sequence is replaced by the target of a different miRNA. The plant transformation vector is then constructed as above, except that it is supplemented by a second chimeric polynucleotide comprising the precursor of the miRNA whose target is used as the trigger sequence. This second chimeric polynucleotide is under the control of the maize histone 2B gene (U.S. Pat. No. 6,177,611). Transformation and monitoring of gene silencing are carried out as above.

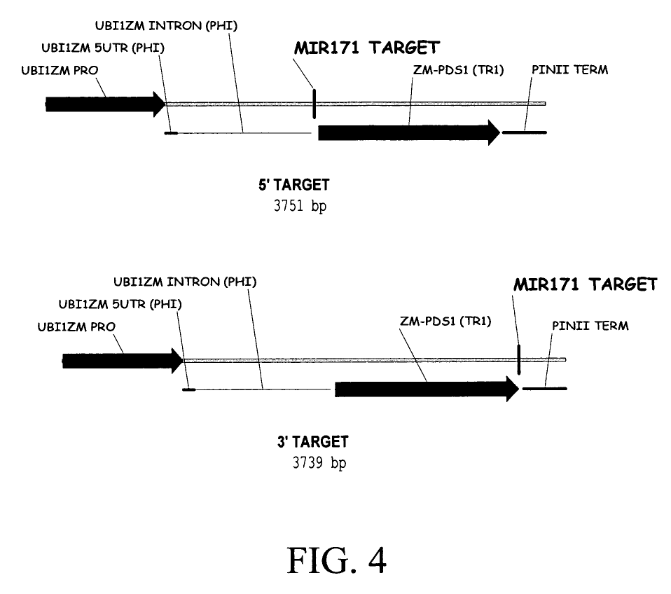

B. The above experiment (example 4.A) is repeated as described, except that as a trigger sequence a sequence complementary to maize miRNA171 (gatattggcacggctcaatca) (SEQ ID NO: 24) is used, such that the trigger sequence is found either 5′ or 3′ of the silencer sequence. See, FIG. 4, for non-limiting examples of such constructs. Transformation is carried out and silencing monitored as described above. The control comprises embryos transformed with a vector having a trigger sequence which has a decreased complementarity to the miRNA.

C. A chimeric polynucleotide is constructed in which the target site for maize miRNA390 is used as trigger sequence and is operably linked to the 3′ end of a silencer sequence. Sequences flanking the trigger and silencer were derived from the ZmTAS3 locus corresponding to the annotated gene PCO085991 (Allen et al. (2005) Cell 121:207-21; Williams et al (2005) PNAS 102: 9703-9708) The silencer sequence comprises a synthetic DNA fragment containing 2 tandem 21 nucleotide segments found in the maize phytoene desaturase gene. Each 21 nucleotide segment is designed to possess the characteristics required for efficient incorporation of a complementary strand into RISC as described by Khvorova et al. ((2003) Cell 115: 199-208) and Schwarz et al. ((2003) Cell 115: 209-216). The unmodified ZmTAS3 sequence is shown in SEQ ID NO:17 and the engineered ZmTAS3 locus designed to silence PDS is shown in SEQ ID NO:18. This combination is operably linked to the maize ubiquitin promoter (Christensen et al. (1989) Plant Mol. Biol. 12:619-632; Christensen et al. (1992) Plant Mol Biol 18:675-689). The resulting chimeric construct, comprising the ubiquitin promoter operably linked to a polynucleotide comprising a fragment of the PDS gene linked to the target of the chosen miRNA as trigger sequence is inserted into a standard vector for maize transformation and includes the bar gene as a selectable marker. The construct is transformed into maize using the procedure described in Example 6. The plants are regenerated to the plantlet stage. Silencing of the PDS gene is monitored by looking for white plantlets. Silencing of PDS interferes with carotenoid biosynthesis and results in bleaching of green tissue under high light conditions. As a control a completely parallel experiment is carried out exactly as described above except that the trigger sequence is altered such that complementarity to miRNA390 is reduced.

SEQ ID NO:17 corresponds to ZmTAS3.PCO085991 which is a 903 nucleotides. The mir390 target sequence corresponds to bases 699-719, the ta-siRNA that target ARF2/3/4 corresponds to bases 543-563 and 564-584.

SEQ ID NO:18 is the modified ZmTAS3 used to silence PDS. The mir390 target sequence corresponds to bases 699-719, the sequence complementary to a synthetic ta-siRNA that targets PDS corresponds to bases 543-563 and 564-584.

### Example 5

### Silencing Using Trigger Sequences in Fungi

In order to provide trigger sequences, miRNAs active in Colletotrichum graminicola (Cg) are cloned and characterized as follows: RNA is prepared from fungal cultures. The size fractionated sRNAs are ligated to 3′ and 5′ RNA-DNA adaptors, PCR amplified using adaptor-specific primers and cloned into plasmid vectors using standard procedures (Llave et al. (2002) Plant Cell 14, 1605-1619). Abundant sRNAs are identified from the sequence analysis of the cloned sRNAs and their complementary nucleotide sequence is incorporated as the trigger element of chimeric constructs as described below.

A chimeric construct comprising the following is constructed:

1. the promoter of the Magnaporthe grisea ribosomal protein 27 promoter (GenBank AY142483; Bourett et al. (2002) Fungal Genet Biol. 37:211-220)

2. as silencer sequence, a sequence containing fragments of both the CgALB1 gene (which encodes a polyketide synthase responsible for production of the black pigment melanin in mycelium) and the CgMES1 gene (which encodes a membrane protein required for hyphal polarization which when silenced produces compact instead of spreading colony morphology). The silencer sequence is SEQ ID NO:13.

3. as trigger, a sequence complementary to one of the miRNAs isolated in the steps outlined above.

This chimeric construct is inserted in a standard transformation vector based on pSM565 (GenBank AY142483; Bourett et al. (2002) Fungal Genet Biol. 37:211-220), which contains a hygromycin resistance gene cassette as a selectable marker. The vector is transformed into Cg protoplasts using standard methods (Thon et al. (2002) MPMI 15:120-128). Silencing of the two genes is monitored by examining colony morphology and color relative to controls. The latter are created using identical procedures and constructs, except that the trigger sequence will be altered to remove complementarity with the miRNA.

### Example 6

### Agrobacterium-Mediated Transformation of Maize and Regeneration of Transgenic Plants

Maize may be transformed with any of the polynucleotide constructs described in Example 4 using the method of Zhao (U.S. Pat. No. 5,981,840, and PCT patent publication WO98/32326). Briefly, immature embryos are isolated from maize and the embryos contacted with a suspension of Agrobacterium, where the bacteria are capable of transferring the polynucleotide construct to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is performed. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The callus is then regenerated into plants (step 5: the regeneration step), and calli grown on selective medium are cultured on solid medium to regenerate the plants.

In specific embodiments, an endosperm culturing system can also be used to suppress expression of sequences in the endosperm. See, for example, U.S. Patent Application 2006/0123518, filed Nov. 30, 2005, entitled “Methods for Culturing Cereal Endosperm”, herein incorporated by reference in its entirety. Agrobacterium-based transformation (or particle bombardment) can also be used when employing this technique. In such embodiments, the sRNAs (for the trigger sequence being used) is present in the endosperm and/or aleurone cells or exogenous sequences are expressed in these tissues.

### Example 7

### Transformation and Fatty Acid Analysis of Somatic Soybean Embryo Cultures

Mature somatic soybean embryos are a good model for zygotic embryos. While in the globular embryo state in liquid culture, somatic soybean embryos contain very low amounts of triacylglycerol or storage proteins typical of maturing, zygotic soybean embryos. At this developmental stage, the ratio of total triacylglyceride to total polar lipid (phospholipids and glycolipid) is about 1:4, as is typical of zygotic soybean embryos at the developmental stage from which the somatic embryo culture is initiated. At the globular stage as well, the mRNAs for the prominent seed proteins, α′-subunit of β-conglycinin, kunitz trypsin inhibitor 3, and seed lectin are essentially absent. Upon transfer to hormone-free media to allow differentiation to the maturing somatic embryo state, triacylglycerol becomes the most abundant lipid class. As well, mRNAs for α′-subunit of β-conglycinin, kunitz trypsin inhibitor 3 and seed lectin become very abundant messages in the total mRNA population. On this basis, somatic soybean embryo system behaves very similarly to maturing zygotic soybean embryos in vivo, and is therefore a good and rapid model system for analyzing the phenotypic effects of modifying the expression of genes in the fatty acid biosynthesis pathway. Most importantly, the model system is also predictive of the fatty acid composition of seeds from plants derived from transgenic embryos.

A. Culture Conditions

Soybean embryogenic suspension cultures (cv. Jack) are maintained in 35 ml liquid medium SB196 (see recipes below) on rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 ml of fresh liquid SB196 (the preferred subculture interval is every 7 days).

Soybean embryogenic suspension cultures are transformed with the plasmids and DNA fragments described in the following examples by the method of particle gun bombardment (Klein et al. (1987) Nature, 327:70). A DuPont Biolistic PDS1000/HE instrument (helium retrofit) is used for all transformations.

B. Soybean Embryogenic Suspension Culture Initiation

Soybean cultures are initiated twice each month with 5-7 days between each initiation. Pods with immature seeds from available soybean plants 45-55 days after planting are picked, removed from their shells and placed into a sterilized magenta box. The soybean seeds are sterilized by shaking them for 15 minutes in a 5% Clorox solution with 1 drop of ivory soap (95 ml of autoclaved distilled water plus 5 ml Clorox and 1 drop of soap). Mix well. Seeds are rinsed using 2 1-liter bottles of sterile distilled water and those less than 4 mm are placed on individual microscope slides. The small end of the seed is cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB196 liquid media for 7 days.

C. Preparation of DNA for Bombardment

A intact plasmid or a DNA plasmid fragment containing the genes of interest as described in Example 3 and the selectable marker gene is used for bombardment. Plasmid DNA for bombardment is routinely prepared and purified using the method described in the Promega™ Protocols and Applications Guide, Second Edition (page 106).

A 50 μl aliquot of sterile distilled water containing 3 mg of gold particles (3 mg gold) is added to 5 μl of a 1 μg/μl DNA solution (intact plasmid prepared as described above), 50 μl 2.5M CaCl₂ and 20 μl of 0.1 M spermidine. The mixture is shaken 3 min on level 3 of a vortex shaker and spun for 10 sec in a bench microfuge. After a wash with 400 μl 100% ethanol the pellet is suspended by sonication in 40 μl of 100% ethanol. Five μl of DNA suspension is dispensed to each flying disk of the Biolistic PDS1000/HE instrument disk. Each 5 μl aliquot contained approximately 0.375 mg gold per bombardment (i.e. per disk).

D. Tissue Preparation and Bombardment with DNA

Approximately 150-200 mg of 7 day old embryonic suspension cultures are placed in an empty, sterile 60×15 mm petri dish and the dish covered with plastic mesh. Tissue is bombarded 1 or 2 shots per plate with membrane rupture pressure set at 1100 PSI and the chamber evacuated to a vacuum of 27-28 inches of mercury. Tissue is placed approximately 3.5 inches from the retaining/stopping screen.

E. Selection of Transformed Embryos

Transformed embryos are selected using hygromycin.

F. Hygromycin (HPT) Selection

Following bombardment, the tissue is placed into fresh SB196 media and cultured as described above. Six days post-bombardment, the SB196 is exchanged with fresh SB196 containing a selection agent of 30 mg/L hygromycin. The selection media is refreshed weekly. Four to six weeks post selection, green, transformed tissue may be observed growing from untransformed, necrotic embryogenic clusters. Isolated, green tissue is removed and inoculated into multiwell plates to generate new, clonally propagated, transformed embryogenic suspension cultures.

G. Embryo Maturation

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 uE/m2s. After this time embryo clusters are removed to a solid agar media, SB166, for 1-2 weeks. Clusters are then subcultured to medium SB103 for 3 weeks. During this period, individual embryos can be removed from the clusters and screened for alterations in their fatty acid compositions as described below. It should be noted that any detectable phenotype, resulting from the expression of the genes of interest, could be screened at this stage. This would include, but not be limited to, alterations in fatty acid profile, protein profile and content, carbohydrate content, growth rate, viability, or the ability to develop normally into a soybean plant.

H. Media Recipes
SB 196 - FN Lite liquid proliferation medium (per liter) -

| | | |
|---|---|---|
| MS FeEDTA - 100× Stock 1 | 10 | ml |
| MS Sulfate - 100× Stock 2 | 10 | ml |
| FN Lite Halides - 100× Stock 3 | 10 | ml |
| FN Lite P, B, Mo - 100× Stock 4 | 10 | ml |
| B5 vitamins (1 ml/L) | 1.0 | ml |
| 2,4-D (10 mg/L final concentration) | 1.0 | ml |
| KNO3 | 2.83 | gm |
| (NH4)2SO4 | 0.463 | gm |
| Asparagine | 1.0 | gm |
| Sucrose (1%) | 10 | gm |
| pH 5.8 | | |

FN Lite Stock Solutions

| Stock # | | 1000 | ml | 500 | ml |
|---|---|---|---|---|---|
| 1 | MS Fe EDTA 100× Stock | | | | |
| | Na₂ EDTA* | 3.724 | g | 1.862 | g |
| | FeSO₄—7H₂O | 2.784 | g | 1.392 | g |
| 2 | MS Sulfate 100× stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—5H₂O | 0.0025 | g | 0.00125 | g |
| 3 | FN Lite Halides 100× Stock | | | | |
| | CaCl₂—2H₂O | 30.0 | g | 15.0 | g |
| | KI | 0.083 | g | 0.0715 | g |
| | CoCl₂—6H₂O | 0.0025 | g | 0.00125 | g |
| 4 | FN Lite P, B, Mo 100× Stock | | | | |
| | KH₂PO₄ | 18.5 | g | 9.25 | g |
| | H₃BO₃ | 0.62 | g | 0.31 | g |
| | Na₂MoO₄—2H₂O | 0.025 | g | 0.0125 | g |

*Add first, dissolve in dark bottle while stirring

SB1 Solid Medium (Per Liter)—

1 pkg. MS salts (Gibco/BRL—Cat# 11117-066)
1 ml B5 vitamins 1000× stock
31.5 g sucrose
2 ml 2,4-D (20 mg/L final concentration)
pH 5.7
8 g TC agar
   SB166 Solid Medium (Per Liter)—

1 pkg. MS salts (Gibco/BRL—Cat# 11117-066)

1 ml B5 vitamins 1000× stock

60 g maltose

750 mg MgCl2 hexahydrate

5 g activated charcoal

pH 5.7

2 g gelrite

SB103 Solid Medium (Per Liter)—

1 pkg. MS salts (Gibco/BRL—Cat# 11117-066)

1 ml B5 vitamins 1000× stock

60 g maltose

750 mg MgCl2 hexahydrate

pH 5.7

2 g gelrite

B. SB 71-4 Solid Medium (Per Liter)—

1 bottle Gamborg's B5 salts w/sucrose (Gibco/BRL—Cat# 21153-036)

pH 5.7

5 g TC agar

### 2,4-D Stock

obtained premade from Phytotech cat# D 295—concentration is 1 mg/ml

B5 Vitamins Stock (per 100 ml)—store aliquots at −20 C

10 g myo-inositol
100 mg nicotinic acid
100 mg pyridoxine HCl
1 g thiamine
If the solution does not dissolve quickly enough, apply a low level of heat via the hot stir plate.
   Chlorsulfuron Stock

1 mg/ml in 0.01 N Ammonium Hydroxide

I. Fatty Acid Analysis of Somatic Soybean Embryo Cultures

Fatty acid methyl esters are prepared from single, matured, somatic soy embryos by transesterification. Embryos are placed in a vial containing 50 μL of trimethylsulfonium hydroxide (TMSH) and 0.5 mL of hexane and are incubated for 30 minutes at room temperature while shaking. Fatty acid methyl esters (5 μL injected from hexane layer) are separated and quantified using a Hewlett-Packard 6890 Gas Chromatograph fitted with an Omegawax 320 fused silica capillary column (Supelco Inc., Cat#24152). The oven temperature is programmed to hold at 220° C. for 2.7 min, increase to 240° C. at 20° C./min and then hold for an additional 2.3 min. Carrier gas is supplied by a Whatman hydrogen generator. Retention times are compared to those for methyl esters of standards commercially available (Nu-Chek Prep, Inc. catalog #U-99-A).

### Example 8

### Silencing Using Trigger Sequences Attached to Synthetic Arrays of 21mers

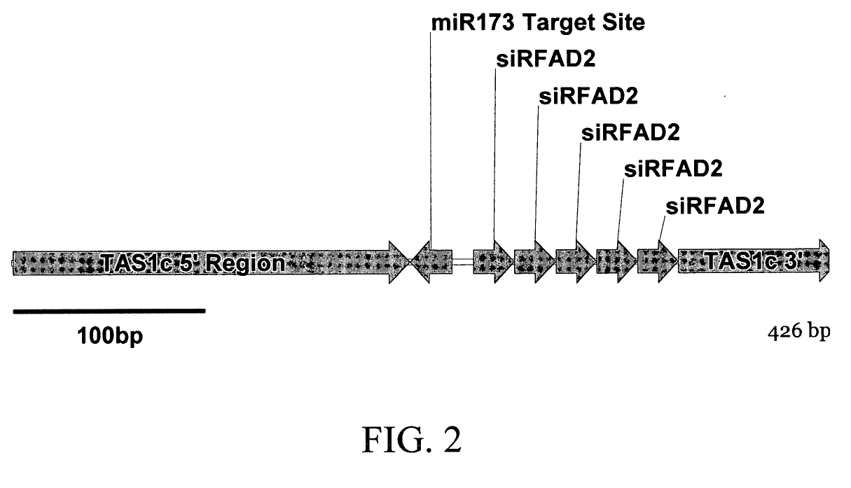

A chimeric polynucleotide was constructed in which the target site for Arabidopsis miRNA (miR173; Allen et al. (2005) Cell 121:207-21) was used as trigger sequence and was operably linked to the 5′ end of a silencer sequence. The silencer sequence comprised a synthetic DNA fragment containing 5 repeated copies of a 21 nucleotide segments complementary to the Arabidopsis fatty acid desaturase 2 (FAD2) gene with the sequence [TTGCTTTCTTCAGATCTCCCA] (SEQ ID NO:14). The trigger sequence complementary to miR173 was followed by 11 nucleotides such that the miR173 cleavage site was separated by 21 nucleotides from the first of the 21 nucleotide FAD2 segments. Sequences flanking the trigger and silencer were derived from the TAS1c locus (Allen et al. (2005) Cell 121:207-21). The chimeric construct is SEQ ID NO:15 and is shown schematically in FIG. 2. The miR173 target site is from nucleotides 205 to 227 of SEQ ID NO:15 and the multimer of FAD2 siRNA is from nucleotides 239 to 344 of SEQ ID NO:15. The 35S promoter and leader sequence (Odell (1985) Nature 313: 810-812) were attached to the 5′ end of the chimeric construct and the phaseolin transcriptional terminator (Barr et al. (2004) Molecular Breeding 13: 345-356) to the 3′ end. The entire chimeric polynucleotide, called FAD2TASwt, was inserted into the standard binary vector pBE851 (Aukerman and Sakai (2003) Plant Cell 15:2730-41) and transformed into Arabidopsis using the method of Clough and Bent (1998) Plant Journal 16:735-43. As a control, the exact same construct was made but with 3 nucleotides of the miR173 target site mutated, as in SEQ ID NO: 16 (referred to as FAD2TASmut). The mutated miR173 target site is from nucleotides 205 to 227 in SEQ ID NO: 16 and the multimer of FAD2 siRNA is from nucleotides 239 to 344 in SEQ ID NO: 16. Transgenic plants containing the experimental construct were monitored for silencing of the FAD2 gene using fatty acid analysis (Browse et al. (1986) Analytical Biochemistry 152: 141-145) and compared to control plants.

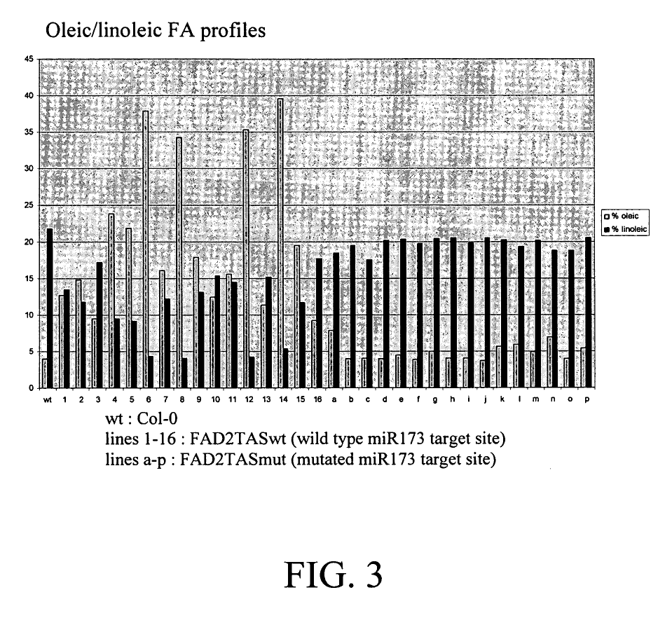

The results are shown in FIG. 3. As can be seen in the figure, lines carrying the experimental construct with the correct trigger sequence virtually all have increased levels of high oleic acid, as would be expected when FAD2 is silenced. This is not seen in the control plants (those designated with letters instead of numbers) where the trigger sequence is not homologous to miR173, nor is it seen in an untransformed plant (wt=wild type).

### Example 9

### Silencing Using Trigger Sequences in Arabidopsis

A. A chimeric polynucleotide is constructed in which the target site for Arabidopsis miRNA 390 is used as trigger sequence and is operably linked to the 3′ end of a silencer sequence. Sequences flanking the trigger and silencer were derived from the TAS3 locus corresponding to the annotated gene At3g17185 (Allen et al. (2005) Cell 121:207-21; Williams et al. (2005) PNAS 102: 9703-9708). The silencer sequence comprises a synthetic DNA fragment containing 2 tandom 21 nucleotide segments found in the Arabidopsis fatty acid desaturase 2 (FAD2) gene. Each 21 nucleotide segment is designed to possess the characteristics required for efficient incorporation of a complementary strand into RISC as described by Khvorova et al. ((2003) Cell 115: 199-208) and Schwarz et al. ((2003) Cell 115: 209-216). The unmodified TAS3 sequence is shown in SEQ ID NO:19 and the engineered TAS3 locus designed to silence FAD2 is shown in SEQ ID NO:20. The 35S promoter and leader sequence (Odell (1985) Nature 313: 810-812) are attached to the 5′ end of the chimeric construct and the phaseolin transcriptional terminator (Barr et al. (2004) Molecular Breeding 13: 345-356) to the 3′ end. The entire chimeric polynucleotide is inserted into a standard binary vector and transformed into Arabidopsis. Transgenic plants containing the experimental construct are monitored for silencing of the FAD2 gene using fatty acid analysis (Browse et al. (1986) Analytical Biochemistry 152: 141-145) and compared to control plants. The latter are created in an identical way except that the trigger sequence is mutated to remove homology to mir390.

SEQ ID NO:19 comprises At3g17185/TAS3 which encompassing Exon 2. The mir390 target sequence corresponds to bases 347-367, the ta-siRNA that targets ARF2/3/4 corresponds to bases 190-209 and 210-230.

SEQ ID NO:20 comprises the Modified TAS3 used to silence FAD2. The mir390 target sequence corresponds to bases 347-367, the sequences complementary to FAD2 targeting ta-siRNA correspond to bases 190-209 and 210-230.

B. A chimeric polynucleotide is constructed in which the target site for Arabidopsis miRNA (miR173; Allen et al. (2005) Cell 121:207-21) was used as trigger sequence and was operably linked to the 5′ end of a silencer sequence. The silencer sequence comprised a fragment of TAS1c where synthetic 21 nt sequences that direct the production of ta-siRNA that silence FAD2 and AP1 replaced endogenous ta-siRNA. The sequence of the endogenous TAS1c locus as well as the modified locus to silence FAD2 and AP1 are shown in SEQ ID NO:21. Transgenic plants containing the experimental construct are monitored for silencing of the FAD2 gene using fatty acid analysis and for silencing of the AP1 gene by visual inspection of floral morphology.

SEQ ID NO:21 comprises a modified TAS1c to silence both FAD2 and AP1. The mir173 target sequence corresponds to bases 367-388, the sequence complementary to a synthetic ta-siRNA that targets FAD2 corresponds to bases 400-420 and the sequence complementary to a synthetic ta-siRNA that targets AP1 corresponds to bases 463-483.

C. A chimeric polynucleotide is constructed in which the target site for Arabidopsis miRNA (miR173; Allen et al. (2005) Cell 121:207-21) is used as trigger sequence and is operably linked to the 5′ end of a silencer sequence. The silencer sequence comprises a modified TAS1c transcript containing a 210 nt region of FAD2. Shown in SEQ ID NO:22. The 35S promoter and leader sequence (Odell (1985) Nature 313: 810-812) are attached to the 5′ end of the chimeric construct and the phaseolin transcriptional terminator (Barr et al. (2004) Molecular Breeding 13: 345-356) to the 3′ end. The entire chimeric polynucleotide is inserted into a standard binary vector and transformed into Arabidopsis. Transgenic plants containing the experimental construct are monitored for silencing of the FAD2 gene using fatty acid analysis (Browse et al. (1986) Analytical Biochemistry 152: 141-145) and compared to control plants. The latter are created in an identical way except that the trigger sequence is mutated to remove homology to miR173.

SEQ ID NO:22 comprises the modified TAS1c to silence FAD2 using a gene fragment. The mir173 target sequence corresponds to bases 367-388, 210 base sequence from FAD2 corresponds to bases 400-609.

D. A chimeric polynucleotide is constructed in which the target site for a synthetic miRNA is used as a trigger sequence. The mutated mir173 (as discussed in Example 8) is used as a trigger sequence and was operably linked to the 5′ end of a silencer sequence. The silencer sequence comprises a synthetic DNA fragment containing 5 repeated copies of a 21 nucleotide segments complementary to the Arabidopsis fatty acid desaturase 2 (FAD2) gene, as disclosed in Example 8. Lines carrying this construct were transformed with a second transgene that expressed a synthetic miRNA complementary to the mutated mir173 trigger sequence. The resulting double transgenic plants are monitored for silencing of the FAD2 gene using fatty acid analysis and compared to control plants.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A chimeric polynucleotide comprising a trigger sequence operably linked to a heterologous silencer sequence of an endogenous target polynucleotide, wherein expression of said chimeric polynucleotide in a cell reduces the level of expression the endogenous target polynucleotide.

2. The chimeric polynucleotide of claim 1, wherein said polynucleotide is operably linked to a promoter.

3. The chimeric polynucleotide of claim 1, wherein said trigger sequence is 5′ or 3′ to the silencer sequence.

4. The chimeric polynucleotide of claim 1, wherein the silencer sequence is orientated to produce a sense or an anti-sense transcript of the native target polynucleotide.

5. The chimeric polynucleotide of claim 1, wherein the silencer sequence comprises at least 19 nucleotides and shares at least 95% sequence identity or at least 95% sequence complementarity to the transcript of the native target polynucleotide.

6. The chimeric polynucleotide of claim 1, wherein said trigger sequence shares at least 78% sequence complementarity to a miRNA or a siRNA.

7. The chimeric polynucleotide of claim 1, wherein said native target polynucleotide is found in a pest.

8. The chimeric polynucleotide of claim 1, wherein the chimeric polynucleotide further comprises a nucleotide sequence comprising a sRNA of the trigger sequence.

9. The chimeric polynucleotide of claim 8, wherein said nucleotide sequence of the sRNA comprises a miRNA.

10. The chimeric polynucleotide of claim 9, wherein said miRNA comprises a pre-miRNA or a primary-miRNA.

11. The chimeric polynucleotide of claim 8, wherein said nucleotide sequence of the sRNA comprises an siRNA.

12. The chimeric polynucleotide of claim 8, wherein said nucleotide sequence comprising the sRNA is operably linked to a second promoter.

13. The chimeric polynucleotide of claim 1, wherein said endogenous target polynucleotide is a native sequence.

14. The chimeric polynucleotide of claim 1, wherein said chimeric polynucleotide comprises at least one structural element of a trans-acting siRNA (TAS) encoding locus.

15. The chimeric polynucleotide of claim 14, wherein said polynucleotide comprising the TAS encoding locus is selected from the group consisting of
a) a polynucleotide set forth in SEQ ID NO: 24, 25, 26, 27, 19, 17, or 28; and,
b) a polynucleotide having at least 90% sequence identity to SEQ ID NO: 24, 25, 26, 27, 19, 17, or 28;
wherein at least one of a TAS ta-siRNA sequence is replaced with said heterologous silencer sequence, and the expression of said chimeric polynucleotide in a cell reduces the level of expression the endogenous target polynucleotide.

16. The chimeric polynucleotide of claim 15, wherein at least one TAS miRNA target site is replaced with at least one heterologous trigger element and at least one of the TAS ta-siRNA sequences is replaced with the heterologous silencer sequence.

17. A vector comprising the chimeric polynucleotide of claim 1.

18. A cell comprising the chimeric polynucleotide of claim 1.

19. The cell of claim 18, wherein said chimeric polynucleotide is stably incorporated into the genome of the cell.

20. The cell of claim 18, wherein said cell is from a eukaryotic organism, a fungi, an animal, or a plant.

21. The cell of claim 20, wherein said plant cell is a monocotyledonous plant cell.

22. The cell of claim 21, wherein said monocotyledonous cell is from maize, barley, millet, wheat or rice.

23. The cell of claim 20, wherein said plant cell is a dicotyledonous plant cell.

24. The cell of claim 23, wherein said dicotyledonous plant cell is from soybean, canola, alfalfa, sunflower, safflower, tobacco, Arabidopsis, or cotton.

25. A plant having the chimeric polynucleotide of claim 1.

26. The plant of claim 25, wherein said chimeric polynucleotide is stably integrated into the genome of the plant.

27. A seed having stably incorporated into its genome the chimeric polynucleotide of claim 1.

28. The seed of claim 27, wherein said seed is from a monocotyledonous plant.

29. The seed of claim 28, wherein said monocotyledonous plant is maize, barley, millet, wheat or rice.

30. The seed of claim 27, wherein said plant is from a dicotyledonous plant.

31. The seed of claim 30, wherein said dicotyledonous plant is soybean, canola, alfalfa, sunflower, safflower, tobacco, Arabidopsis, or cotton.

32. A grain having the chimeric polynucleotide of claim 1.

33. A method for reducing the level of expression of a target polynucleotide of interest comprising
a) introducing into a cell a chimeric polynucleotide comprising a trigger sequence operably linked to a heterologous silencer sequence of an endogenous target polynucleotide; and,
b) expressing said chimeric polynucleotide.

34. The method of claim 33, wherein said trigger sequence is positioned 5′ or 3′ to the silencer sequence.

35. The method of claim 33, wherein the silencer sequence is oriented to produce the sense or the anti-sense sequence of the target polynucleotide.

36. The method of claim 33, wherein the silencer sequence comprises at least 19 nucleotides and shares at least 95% sequence identity or at least 95% sequence complementarity to the transcript of the endogenous target polynucleotide.

37. The method of claim 33, wherein said trigger sequence shares at least 78% sequence complementarity to an endogenous miRNA or a siRNA.

38. The method of any one of claims 33, wherein said cell is in an organism and said endogenous target polynucleotide is from a pest of said organism.

39. The method of claim 33, wherein said chimeric polynucleotide is stably incorporated into the genome of the cell.

40. The method of claim 33, wherein said cell is from a eukaryotic organism, a fungi, or an animal.

41. The method of claim 33, wherein said cell is from a plant.

42. The method of claim 41, wherein said plant cell is a monocotyledonous plant cell.

43. The method of claim 42, wherein said monocotyledonous plant cell is from maize, barley, millet, wheat or rice.

44. The method of claim 41, wherein said plant cell is a dicotyledonous plant cell.

45. The method of claim 44, wherein said dicotyledonous plant cell is from soybean, canola, alfalfa, sunflower, safflower, tobacco, Arabidopsis, or cotton.

46. The method of claim 41, wherein reducing the level of the target polynucleotide modulates the fatty acid composition of the plant.

47. The method of claim 46, wherein the modulation of the fatty acid composition comprises an increase in the level of oleic acid in a seed of the plant.

48. The method of claim 46, wherein reducing the level of the target polynucleotide modulates the level of a storage protein.

49. The method of claim 46, wherein reducing the level of the target polynucleotide modulates glycinin.

50. The method of claim 33, wherein said chimeric polynucleotide comprises at least one structural element of a trans-acting siRNA (TAS) encoding locus.

51. The chimeric polynucleotide of claim 50, wherein said polynucleotide comprising a TAS encoding locus selected from the group consisting of
a) a polynucleotide set forth in SEQ ID NO: 24, 25, 26, 27, 19, 17, or 28; and,
b) a polynucleotide having at least 90% sequence identity to SEQ ID NO: 24, 25, 26, 27, 19, 17, or 28;
wherein at least one TAS ta-siRNA sequence is replaced with said heterologous silencer sequence; and the expression of said chimeric polynucleotide in a cell reduces the level of expression the endogenous target polynucleotide.

52. The chimeric polynucleotide of claim 51, wherein at least one TAS miRNA target site is replaced with a heterologous trigger sequence and at least one TAS ta-siRNA sequence is replaced with said heterologous silencer sequence.

53. An isolated polynucleotide selected from the group consisting of:
a. the polynucleotide set forth in SEQ ID NO: 28;
b. the polynucleotide having at least 90% sequence identity to the sequence set forth in SEQ ID NO:28, wherein said polynucleotide retains the ability to reduce the level of a target polynucleotide; and,
c. the polynucleotide having at least 50 consecutive nucleotides of SEQ ID NO:28, wherein said polynucleotide retains the ability to reduce the level of a target polynucleotide.

54. A transgenic plant or plant cell having a heterologous polynucleotide of claim 53.

55. A transgenic seed from the plant of claim 54.


SEQUENCE LISTING (28 sequences)

SEQ ID NO: 1 - DNA, 21 bp, Arabidopsis thaliana
  misc_feature (0)...(0): miRNA159
uuuggauugaagggagcucua

SEQ ID NO: 2 - DNA, 21 bp, Arabidopsis thaliana
  misc_feature (0)...(0): miRNA161
uugaaagugacuacaucgggg

SEQ ID NO: 3 - DNA, 21 bp, Arabidopsis thaliana
  misc_feature (0)...(0): miRNA165
ucggaccaggcuucauccccc

SEQ ID NO: 4 - DNA, 21 bp, Arabidopsis thaliana
  misc_feature (0)...(0): miRNA168
ucgcuuggugcaggucgggaa

SEQ ID NO: 5 - DNA, 21 bp, Oryza sativa
  misc_feature (0)...(0): miRNA168
ucgcuuggugcagaucgggac

SEQ ID NO: 6 - DNA, 32 bp, Artificial Sequence
  oligonucleotide primer
gaattcgcggccgcccaatctattgggttctc

SEQ ID NO: 7 - DNA, 32 bp, Artificial Sequence
  oligonucleotide primer
gaattcgcggccgcgagtgtgacgagaagaga

SEQ ID NO: 8 - DNA, 26 bp, Artificial Sequence
  oligonucleotide primer
gccaaggaaagcgtgaacaagaccag

SEQ ID NO: 9 - DNA, 30 bp, Artificial Sequence
  oligonucleotide primer
tgtggcacgaacattcatattgggcactga

SEQ ID NO: 10 - DNA, 30 bp, Artificial Sequence
  oligonucleotide primer
tcagtgcccaatatgaatgttcgtgccaca

SEQ ID NO: 11 - DNA, 26 bp, Artificial Sequence
  oligonucleotide primer
gttctttatctgcctggcctgctggc

SEQ ID NO: 12 - DNA, 1361 bp, Zea mays
  misc_feature (0)...(0): fragment of phytoene desaturase
ctagccagacaagatcttttgcggggcaacttcctcctcagagatgttttgcgagtagtcactatacaagctttgccgtgaaaaaacttgtctcaaggaataaaggaaggagatcacaccgtagacatcctgccttgcagttgtctgcaaaggattttccaagtcctccactagaaagcacaataaactatttggaagctggacagctctcttcattttttagaaacagcgaacgccccagtaagccgttgcaggtcgtggttgctggtgcaggattggctggtctatcaacagcgaagtatctggcagatgctggacataaacccatattgcttgaggcaagagatgttttgggtggaaaggtagctgcttggaaggatgaagatggagattggtacgagactgggcttcatatcttttttggagcttatcccaacatacagaatctgtttggcgagcttaggattgaggatcgtttacagtggaaagaacactctatgatattcgccatgccaaacaagccaggagaattcagccggtttgatttcccagaaactttgccagcacctataaatgggatatgggccatattgagaaacaatgaaatgcttacctggcccgagaaggtgaagtttgcaatcggacttctgccagcaatggttggtggtcaaccttatgttgaagctcaagatggcttaaccgtttcagaatggatgaaaaagcagggtgttcctgatcgggtgaacgatgaggtttttattgcaatgtccaaggcactcaatttcataaatcctgatgagctatctatgcagtgcattttgattgctttgaaccgatttcttcaggagaagcatggttctaaaatggcattcttggatggtaatccgcctgaaaggctatgcatgcctattgttgatcacattcggtctaggggtggagaggtccgcctgaattctcgtattaaaaagatagagctgaatcctgatggaactgtaaaacacttcgcacttagtgatggaactcagataactggagatgcttatgtttgtgcaacaccagtcgatatcttcaagcttcttgtacctcaagagtggagtgaaattacttatttcaagaaactggagaagttggtgggagttcctgttatcaatgttcatatatggtttgacagaaaactgaacaacacatatgaccaccttcttttcagcaggagttcacttttaagtgtctatgcagacatgtcagtaacctgcaaggaatactatgacccaaaccgttcaatgctggagttggtctttgctcctgcagacgaatggattggtcgaagtgacactgaaatcatcgatgcaactatggaagag

SEQ ID NO: 13 - DNA, 600 bp, Artificial Sequence
  silencer sequence comprising fragments of CgALB1 and CgMES1
gtacggatgttggatcgagaaccctggctacttcgatcccaggttcttcaacatgtctccccgtgaggccttccagaccgatcccatgcagcgtatggctttgaccaccgcctacgaggcgttggagatgtgcggctatgtccccaacaggacgccctctacaaagctggaccgtatcggtaccttctacggccagacttctgacgattggcgtgaaatcaacgctgcccaagaagtcgacacctactacatcactggtggtgtgcgagctttcggccctggtcgcatcaactaccacttattcgcaaaagctacctggcgtttccagagcagggacaaaagctcatattgaggggtcgtcacaatactcggtaccccgcgatacgcacgagttcgaaagcaaagtcatgtacaagggcatcccgattcctatcaaggttcctgttgccgtcatgcccgaaaccgtaggcgacttctctttgatcaaactcatccagaaattttctgagccgcagggcaaagcgccacagccttttcccctgcacgcgcagttgacaacaaacggcccgaatacacacccgatcattgtgctagtcaacg

SEQ ID NO: 14 - DNA, 21 bp, Arabidopsis thaliana
ttgctttcttcagatctccca

SEQ ID NO: 15 - DNA, 426 bp, Artificial Sequence
  FAD2TASwt chimeric polynucleotide comprising a trigger sequence to miR173 and a silencer sequence comprising 5 repeated copies of a 21 nucleotide segment complementary to the Arabidopsis FAD2 gene.
catagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttattgctttcttcagatctcccattgctttcttcagatctcccattgctttcttcagatctcccattgctttcttcagatctcccattgctttcttcagatctcccaaaaacaatgaatattgttttgaatgtgttcaagtaaatgagattttcaagtcgtctaaagaacagttgctaatacagttactta

SEQ ID NO: 16 - DNA, 426 bp, Artificial Sequence
  FAD2TASmut  chimeric polynucleotide comprising a trigger sequence that is not homologous to miR173 and a silencer sequence comprising 5 repeated copies of a 21 nucleotide segment complementary to the Arabidopsis FAD2 gene.
catagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctaaacaagagaatagaccatttattgctttcttcagatctcccattgctttcttcagatctcccattgctttcttcagatctcccattgctttcttcagatctcccattgctttcttcagatctcccaaaaacaatgaatattgttttgaatgtgttcaagtaaatgagattttcaagtcgtctaaagaacagttgctaatacagttactta

SEQ ID NO: 17 - DNA, 903 bp, Zea mays
  misc_feature (0)...(0): ZmTAS3.pc0085991
ccaagccagctagcaactcaaaggaggaagactagaagatatatagccttctccttgtgtgacactcataaataaactgttagcagcttgcgagcactgcaagatctttctgctggcatctgaatagtcagctgctggcatctatccgggcacctctcgaccagccagaggcaggatacctggatcccaaactagctggccaacatggcgaacgatgaccctgtggacgacgaaaaccccagatccaacaccactaataacagcatcaatgacaagttggctgaagtgttcgacatggtctcagtccacaaacatcagtataaaatgccgacgttcgccttccacaccaacaagacgatcaccgaagctttacatcggcctctactgcgagcacaagctagcatggtcacaacctcctagcctatatatgaactggtgttatcccgactgaacttttctttttgttgacacctttcgtcaggcctctccaccagaggttgactcgtatgtctgttttctggtttctcataccagaattaacgttcttgaccttgtaaggcctcttcttgaccttgtaaggctctcactctgtgtctgcatccttctcgcatcccttgtttccttctttcccactacatgcaggatcagtcccgatattgccgtgtttgccagccttctgcatccacctactttctgttcccttctatccctcctgagctaattgttgctcctatgtaatccgtgtttattatttcggtaccatgatgatgaaattagttttaatttattgtaagactagttcagttaatgttttcaccctgtggatgctctgcaatacatgaacatacatatgcaaacactcattcatccatttattttttattaaaaaaaaaaaaaaaaaaa

SEQ ID NO: 18 - DNA, 903 bp, Artificial Sequence
  modified ZmTAS3 to silence PDS
ccaagccagctagcaactcaaaggaggaagactagaagatatatagccttctccttgtgtgacactcataaataaactgttagcagcttgcgagcactgcaagatctttctgctggcatctgaatagtcagctgctggcatctatccgggcacctctcgaccagccagaggcaggatacctggatcccaaactagctggccaacatggcgaacgatgaccctgtggacgacgaaaaccccagatccaacaccactaataacagcatcaatgacaagttggctgaagtgttcgacatggtctcagtccacaaacatcagtataaaatgccgacgttcgccttccacaccaacaagacgatcaccgaagctttacatcggcctctactgcgagcacaagctagcatggtcacaacctcctagcctatatatgaactggtgttatcccgactgaacttttctttttgttgacacctttcgtcaggcctctccaccagaggttgactcgtatgtctgttttctggtttctcataccagaattaacgaaccgtttcagaatggatgaaaaccgtttcagaatggatgaacactctgtgtctgcatccttctcgcatcccttgtttccttctttcccactacatgcaggatcagtcccgatattgccgtgtttgccagccttctgcatccacctactttctgttcccttctatccctcctgagctaattgttgctcctatgtaatccgtgtttattatttcggtaccatgatgatgaaattagttttaatttattgtaagactagttcagttaatgttttcaccctgtggatgctctgcaatacatgaacatacatatgcaaacactcattcatccatttattttttattaaaaaaaaaaaaaaaaaaa

SEQ ID NO: 19 - DNA, 440 bp, Artificial Sequence
  AT3g17185/TAS3 which comprises the TAS3 locus corresponding to the annotated gene At3g17185 further comprising the mir390 target sequence and the ta-siRNA that targets ARF2/3/4.
gcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttct

SEQ ID NO: 20 - DNA, 440 bp, Artificial Sequence
  modified TAS3 to silence FAD2
gcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttcccaaagcagaaatcagcaatcccaaagcagaaatcagcaatctctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttct

SEQ ID NO: 21 - DNA, 990 bp, Artificial Sequence
  modified TAS1c to silence both FAD2 and AP1
aaacctaaacctaaacggctaagcccgacgtcaaataccaaaaagagaaaaacaagagcgccgtcaagctctgcaaatacgatctgtaagtccatcttaacacaaaagtgagatgggttcttagatcatgttccgccgttagatcgagtcatggtcttgtctcatagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttgggagatctgaagaaagcaatattctaagtccaacatagcgtattctaagttcaacatatcgctgtatcaagaagatgatcctacatattccaggatatgcaaaagaaaacaatgaatattgttttgaatgtgttcaagtaaatgagattttcaagtcgtctaaagaacagttgctaatacagttacttatttcaataaataattggttctaataatacaaaacatattcgaggatatgcagaaaaaaagatgtttgttattttgaaaagcttgagtagtttctctccgaggtgtagcgaagaagcatcatctactttgtaatgtaattttctttatgttttcactttgtaattttatttgtgttaatgtaccatggccgatatcggttttattgaaagaaaatttatgttacttctgttttggctttgcaatcagttatgctagttttcttataccctttcgtaagcttcctaaggaatcgttcattgatttccactgcttcattgtatattaaaactttacaactgtatcgaccatcatataattctgggtcaagagatgaaaatagaacaccacatcgtaaagtgaaat

SEQ ID NO: 22 - DNA, 1095 bp, Artificial Sequence
  modified TAS1c to silence FAD2
aaacctaaacctaaacggctaagcccgacgtcaaataccaaaaagagaaaaacaagagcgccgtcaagctctgcaaatacgatctgtaagtccatcttaacacaaaagtgagatgggttcttagatcatgttccgccgttagatcgagtcatggtcttgtctcatagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttaatgggtgcaggtggaagaatgccggttcctacttcttccaagaaatcggaaaccgacaccacaaagcgtgtgccgtgcgagaaaccgcctttctcggtgggagatctgaagaaagcaatcccgccgcattgtttcaaacgctcaatccctcgctctttctcctaccttatcagtgacatcattatagcctcatgcttctactacgtcgccaaaacaatgaatattgttttgaatgtgttcaagtaaatgagattttcaagtcgtctaaagaacagttgctaatacagttacttatttcaataaataattggttctaataatacaaaacatattcgaggatatgcagaaaaaaagatgtttgttattttgaaaagcttgagtagtttctctccgaggtgtagcgaagaagcatcatctactttgtaatgtaattttctttatgttttcactttgtaattttatttgtgttaatgtaccatggccgatatcggttttattgaaagaaaatttatgttacttctgttttggctttgcaatcagttatgctagttttcttataccctttcgtaagcttcctaaggaatcgttcattgatttccactgcttcattgtatattaaaactttacaactgtatcgaccatcatataattctgggtcaagagatgaaaatagaacaccacatcgtaaagtgaaat

SEQ ID NO: 23 - DNA, 21 bp, maize
gatattggcacggctcaatca

SEQ ID NO: 24 - DNA, 930 bp, Arabidopsis thaliana
  misc_feature (0)...(0): TAS1a
ataaacctaaacccctaagcggctaagcctgacgtcatataccaaaaagagtaaacatgagcgccgtcaagctctgcaagtacaatctcatcttaactcaaaagttgagataggttcttagatcaggttccgcctttagatcgagtcatggtcttgtctgatagaaaggtactttcttttacttctcttgattagcgtctatagctagattgagatcgagtttgtgagatgttaggttcgatatccctgtctatttgtcaccagccatgtaggagtttcgtcccttcccctcccgtcgccctctctgtttttggtattcattggaatacggagatatattttcaagaggagaaatattgttttgttgtgatttttctctacaagcgaatgagtcattcatcctaagtccaacatagcgttcgataagatcttagaaaattattttaagtctaacatagcgtttgattggatcttaggaaattattctaagtccaacatagcgtagagaaatggaagatatcgtgaatgatatttgtagtaatggcgaaactagaaaaagcattggatatattctaggatatgcaaaagttatccttgaatatgttcacattaaatgttattttctacttaatgaacagttgatgatacaattattttctttaaaattgtttccgtgtaaccaaaacatatttcagtatatgcaaaataaaaaatggatgttggtattcttattttgcaaggcttgtaatgggtgttgtgtagtctcttttacaaggtgttgtgaagtctacatgaagcaagtcagctaattacatgcatctttcacattgtaattaatttgatttcaattttgtaattttatttgcttttgtgtaccaaagctgaaatcaaattgtttacaatttcaatataaatgatataatttttaca

SEQ ID NO: 25 - DNA, 839 bp, Arabidopsis thaliana
  misc_feature (0)...(0): TAS1b
aaatctaaacctaagcggctaagcctgacgtcatttaacaaaaagagtaaacatgagcgccgtcaagctctgcaactacgatctgtaactccatcttaacacaaaagttgagataggttcttagatcaggttccgctgttaaatcgagtcatggtcttgtctcatagaaaggtactttcttttacttctcttgagtagcttctatagctagattgagattgaggttttgagatattaggttcgatgtcccggtctatttgtcaccagccatgtgtcagtttcgaccagtcccgtgctctctgtatttggttttatcggaatacggagatctattttcaggaggagacaactttgttttcttgtgatttttctcaacaagcgaatgagtcattcatcggtatctaaccattcaccatattatcagagtagttatgattgataggatggtagaagaatattctaagtccaacatagcatattctaagtccaacatagcgtaaaaaattgggagatatccggaatgatattatacgtaaaaaaaaatgggagatgtccggaatgatatttgtaatatttttatgttaacgaaacatattttaggatatgcaaaaaaaagtagatgttggtattcttgttttgcaagatttgtaatgggagttgtgtagtctttttatgatgtgtcatgaagtctaccgccaattacatacatcattcactttgtaattaaattgtcttcaagtttgtaattttatttttgttttatgtaccaaaatctaaattcagttgtttacaacttgataacaaaaaaaaagttatacattacttatgttttcacactc

SEQ ID NO: 26 - DNA, 990 bp, Arabidopsis thaliana
  misc_feature (0)...(0): TAS1c
aaacctaaacctaaacggctaagcccgacgtcaaataccaaaaagagaaaaacaagagcgccgtcaagctctgcaaatacgatctgtaagtccatcttaacacaaaagtgagatgggttcttagatcatgttccgccgttagatcgagtcatggtcttgtctcatagaaaggtactttcgtttacttcttttgagtatcgagtagagcgtcgtctatagttagtttgagattgcgtttgtcagaagttaggttcaatgtcccggtccaattttcaccagccatgtgtcagtttcgttccttcccgtcctcttctttgatttcgttgggttacggatgttttcgagatgaaacagcattgttttgttgtgatttttctctacaagcgaatagaccatttatcggtggatcttagaaaattattctaagtccaacatagcgtattctaagttcaacatatcgacgaactagaaaagacattggacatattccaggatatgcaaaagaaaacaatgaatattgttttgaatgtgttcaagtaaatgagattttcaagtcgtctaaagaacagttgctaatacagttacttatttcaataaataattggttctaataatacaaaacatattcgaggatatgcagaaaaaaagatgtttgttattttgaaaagcttgagtagtttctctccgaggtgtagcgaagaagcatcatctactttgtaatgtaattttctttatgttttcactttgtaattttatttgtgttaatgtaccatggccgatatcggttttattgaaagaaaatttatgttacttctgttttggctttgcaatcagttatgctagttttcttataccctttcgtaagcttcctaaggaatcgttcattgatttccactgcttcattgtatattaaaactttacaactgtatcgaccatcatataattctgggtcaagagatgaaaatagaacaccacatcgtaaagtgaaat

SEQ ID NO: 27 - DNA, 1054 bp, Arabidopsis thaliana
  misc_feature (0)...(0): TAS2
aaacctagtcgtgacgtcaaaaacaaagagaaaaataagtataagcgccgccaagctctgcaagagatcgagaaaagagccactttggtgaaacactatagttgtgttggattcagaggacagaatctcctgtcacactgatgggtttcgaagatcagattcagctgttagattgattctccatcttgtatcccactgaaaggtacttttatagctagtcctttctatgagtagcctatcatagcatcttctatagctttaggttgggtttgggagtgagtttacgagttacaagttggtttaatgataatatcttggatgatacaatggatttgttaccaagcatgtgtcagtcacggctcctcctctctgttttttggtttcactagaataaatacggcggtttacgagttgaaacgacatggttttgtgatttttctctccaagcgaatgatgatacttaaactattcacttgattatagtttgaacttgtgtattttgaaacacgatgttcaatagatttagatggtagttcaagtattccagatggtagaaatgggatatacatatatgtttcagtcttatccccgtaaaaaaagttgtaactcttgttgatcggatggtagaaacataggtctttaatcccatataggtattcgagtatatgcaaaagagtaagatggatcttgataatctttgttttagtaaacatataagattcattttatatcttttgtaatactaaacatattcatggatatgcaaaaagaaaactaggtatatggttgtgtgatgaagaaattacaaaagacatcattgatgtttgaggatatatgtcgaaagtgaagtttttagcaaactatgttgaaagagcattgtgaagcacattaaagagcgttcatcacttttgcacttgtaattttctcggatcattgtatttgtaccttttagtgtagtcttctgttgttgtaatttcattattaataggaaaaattatcttatgttcattattgatacctttcactgtctaatcaaataatcagtttcgttgct

SEQ ID NO: 28 - DNA, 947 bp, Glycine max
  misc_feature (0)...(0): GMTAS3
cacgagccataacaaaaagtcctctctctctctctctctttcacactctcactttttctgtgcttctcttctctctcctttcaccctcttggaataatagagtagggagaaaaaaaagggtctcaaagatgagaccaaaagacgggtttttcatcaaaggtccctataagggagggttttttctcatgcatgtggctagcagcagcagcaagtgcaaggtggccatgagcatgaccatggtcaacgaccctttgaggagaattgggaggtgtgaccaaaaaaaagaagccattagtggctctgaaattggagctgacaattctcctgcagcaggagcagcttcttcattctaatctggtgctatcctatctgagctttttactactactactaccctttctttcttcatctaatttctaccacacttttctctttgtttttcccttggagtcttcttcttgaccttgtaagaccttttcttgaccttgtaagacctcacaccctatctcttctctttgtttttgcttttgtggaagaccctgtatcactatccactgatatagagtttgatctccttctttccccgttaccacccaactcatactttccttccttgtctatccctcctgagctgttccaatttaattaatttggcctaccatatatgatgcaacaatttaatgtaatattatgccataccatggtatcatatggtatggtatagcttcatagaaattagttaaggtaaggtccttaatcttagaagttacaacctctgccaaatgtgttgttagctatttggagaccatggcttccaagagagtacaacaattggggtatattatttttattttatttgatattttttttttatcttggtgagaaattaatgtagttcgtgcagcatcatggttggtcgtggtccttaaggtatctgtggtaagttg